(19) United States
(12) Patent Application Publication
(10) Pub. No.: US 2021/0252256 A1
(43) Pub. Date: Aug. 19, 2021

(54) MICROSTRUCTURES TO ATTACH APPLIANCES TO TISSUES

(71) Applicants: Ronald J. Berenson, Mercer Island, WA (US); Cheuk Yin Paul Leung, Bellevue, WA (US); Nick Rogerson, Seattle, WA (US)

(72) Inventors: Ronald J. Berenson, Mercer Island, WA (US); Cheuk Yin Paul Leung, Bellevue, WA (US); Nick Rogerson, Seattle, WA (US)

(21) Appl. No.: 17/180,217

(22) Filed: Feb. 19, 2021

Related U.S. Application Data
(60) Provisional application No. 62/978,426, filed on Feb. 19, 2020.

(51) Int. Cl. A61M 25/02; A61B 5/145; A61B 5/00; A61B 5/257; A61B 5/28; A61B 5/291; A61F 13/00; A61F 13/02

(52) U.S. Cl. CPC A61M 25/02; A61B 5/14532; A61B 5/68335; A61B 5/257; A61B 5/28; A61B 5/291; A61M 2025/0286; A61F 13/0259; A61B 5/6839; A61B 5/6831; A61M 2025/0246; A61M 2025/0266; A61F 13/00063

(57) ABSTRACT

An attachment device used to attach appliances to tissue can incorporate microstructures. A medical device system can comprise a coupling device comprising a base layer, a coupling attached to an air side of the base layer, a microstructure attached to a skin side of the base layer and a patient interaction feature, and an appliance comprising a structure mounted to the coupling to interface with the patient interaction feature. An appliance device comprises a base layer, an appliance structure attached to an air side of the base layer, and a microstructure attached to a skin side of the base layer. An appliance attachment system comprises an appliance, a first component comprising an adhesive backing containing microstructures that are configured to attach to skin, and a second component configured to attach the appliance to the first component.

### CLAIM OF PRIORITY

This application claims the benefit of U.S. Provisional Patent Application Ser. No. 62/978,426, filed on Feb. 19, 2020, the benefit of priority of which is claimed hereby, and which is incorporated by reference herein in their entirety.

### CROSS-REFERENCE TO RELATED PATENT DOCUMENTS

The present application is related to US Patent Publication Nos. 2015/0305739; and 2017/0333039; the entire contents of which are incorporated herein by reference.

### BACKGROUND

Many medical appliances are attached to the skin, including: 1) therapeutic devices, such as pumps to administer drugs like insulin, analgesics and chemotherapy, 2) diagnostic and monitoring devices such as glucose and EKG monitors, 3) catheters through which fluids containing drugs, minerals, or nutritional molecules such as amino acids, blood or its components, are administered, 4) surgical drains, chest tubes, catheters, ostomy bags and other devices that are used to remove body fluids, such as infectious material, tissue, exudate, blood, fecal materials, and urine, from the body, 5) catheters used for either peritonea dialysis or hemodialysis, 6) EKG and EEG electrodes used for patient monitoring, 7) nasogastric tubes, 8) enterostomy tubes, and 9) facial masks and endotracheal tubes. The most common materials and devices used to attach appliances are adhesives and tapes.

### OVERVIEW OF THE PRESENT DISCLOSURE

The use of adhesives and tapes for the attachment of appliances suffer from several drawbacks that make them suboptimal.

First, secure attachment is a challenge. This can be a problem with initial application, but is more problematic when wearing the appliance. Appliances tend to become dislodged for a variety of reasons.

The most common reason for detachment is due to the presence of moisture that reduces the effectiveness of the adhesive properties of the tape or adhesive. The moisture can come from a variety of sources, including drainage of body fluids, such as blood, exudate, fecal material, or urine. It can also be a result of sweat. Finally, the moisture may come from direct exposure to water during showering, bathing, or swimming. Solutions, ointments, and creams applied to the area in which the appliance is attached can also cause the appliance to fall off of the skin. Detachment can also come from body movement or accidental dislodgement when removing or putting on clothing or using towels that are used to dry the area. Dislodgement is problematic as it can reduce the effectiveness of the device for its intended clinical application. It can also lead to excess bleeding, loss of body fluids, leakage of fecal material or other sources of potential infection. Drainage and bleeding cannot only put patients at risk of infections, but also caregivers who may also be exposed to transmissible microbial agents such as viruses. In some cases, it can lead to life-threatening complications. For example, dislodgement of vascular catheter can cause serious bleeding that can lead to hypovolemia, organ failure, and death. Dislodgement requires more frequent replacement of tapes and adhesives. This not only requires more resources, but increases risks associated with their replacement—pain, skin trauma, infection, and device malfunctions.

Second, tapes and adhesives can be irritating to the skin. They can cause skin damage, especially in fragile skin of elderly, diabetics, chronically ill patients, (e.g., patients on dialysis), and patients on agents that can injure the skin such as steroids. Damage can be manifested by inflammation, but can lead to blisters and compromise of skin integrity in more severe cases. This places patients at risk for infections and potentially skin ulcers.

Third, the tapes and adhesives can be painful during application, while on the skin, or upon their removal from the skin.

Sutures or traditional medical staples are used to attach appliances to the skin. However, they are of limited use due to the complexity of using these products, difficulty in attaching them to appliances, pain and the trauma and its resultant complications associated with their use.

The present inventors have recognized the challenges of tapes and adhesives as well as sutures and staples, and that there is a resulting strong need for new devices that can be used to attach appliances to the skin.

The present inventors have recognized that microstructures and microstructure arrays can be used to facilitate attachment of appliances to skin. In examples, the microstructures can be used to attach appliances directly and indirectly to skin.

In an example, an attachment device used to attach appliances to tissue incorporates microstructures.

In an example, a coupling device for attaching an appliance to a patient can comprise a base layer, a coupling attached to an air side of the base layer, and a microstructure attached to a skin side of the base layer

In an example, a medical device system can comprise a coupling device comprising a base layer, a coupling attached to an air side of the base layer, a microstructure attached to a skin side of the base layer, and a patient interaction feature; and an appliance comprising a structure mounted to the coupling to interface with the patient interaction feature.

In an example, an appliance device can comprise a base layer, an appliance structure attached to an air side of the base layer, and a microstructure attached to a skin side of the base layer.

In an example, an appliance attachment system can comprise an appliance, a first component comprising an adhesive backing containing microstructures that are configured to attach to skin, and a second component configured to attach the appliance to the first component.

In an example, an appliance attachment cover can comprise a cover body, an appliance securing feature incorporated into the cover body, and a microstructure attached to a skin side of the cover body.

In an example, a medical device configured for attaching a vascular catheter to a patient can comprise a base layer, an appliance structure attached to an air side of the base layer, the appliance structure configured to engage a radial arterial catheter, and a microstructure attached to a skin side of the base layer.

In an example, a medical device configured for attaching a radial arterial catheter to a patient can comprise a base layer, an appliance structure attached to an air side of the base layer, the appliance structure configured to engage a radial arterial catheter, and a microstructure attached to a skin side of the base layer.

In an example, a method for coupling an appliance to a patient can comprise positioning a backing of a coupling device adjacent tissue, attaching a microstructure mounted to the backing to the tissue, and attaching an appliance to a coupling feature of the backing.

In an example, a method for coupling an appliance to a patient can comprise positioning a backing of an appliance device adjacent tissue, attaching a microstructure mounted to the backing to the tissue, and engaging an appliance secured to the patient via the backing with the tissue.

### BRIEF DESCRIPTION OF THE DRAWINGS

FIG. 1 is a perspective view of an appliance device using microstructure attachment features for directly coupling an appliance to tissue.

FIG. 2 is a bottom view of the appliance device of FIG. 1 showing a backing, an appliance, a patient interaction feature, a first microstructure array and a second microstructure array.

FIG. 3 is a side view of the appliance device of FIG. 2 showing the first and second microstructure arrays attached to a skin-facing side of the backing and the appliance attached to an air-facing side of the backing.

FIG. 4A is a closeup and side view of a microstructure protruding from a microstructure array attached to the skin-facing side of the backing.

FIG. 4B is an isolated view of a microstructure array of the appliance device of FIGS. 1-4A showing a foundation and microstructures.

FIG. 5 is an exploded view of an appliance device using microstructure attachment features for indirectly coupling an appliance to tissue via a coupler.

FIG. 6 is a top view of the appliance device of FIG. 5 showing a patient interaction feature and a coupler attached to a backing.

FIG. 7 is a bottom view of the appliance device of FIG. 6 showing the patient interaction feature, a microstructure array and an adhesive layer attached to the backing.

FIG. 8 is a side view of the appliance device of FIG. 7 showing the microstructure array attached to a skin-facing side of the backing and the coupler attached to an air-facing side of the backing.

FIG. 9A is a closeup and side view of a microstructure protruding from the microstructure array attached to the skin-facing side of the backing.

FIG. 9B is an isolated view of a microstructure array of the appliance device of FIGS. 5-9A showing a foundation and microstructures.

FIG. 10 is a perspective view of an appliance cover having an integrated protective cover for holding a catheter and a tube in engagement with tissue.

FIG. 11 is a perspective view of the tissue, catheter and tube of FIG. 10 with the appliance cover exploded therefrom.

FIG. 12 is a perspective view of an appliance cover and a separate protective cover with separate backings for holding a catheter and a tube in engagement with tissue.

FIG. 13 is a perspective view of the tissue, catheter and tube of FIG. 12 with the appliance cover, protective dressing and separate backings exploded therefrom.

FIG. 14 is a perspective view of an appliance cover and a separate protective cover with an integrated backing for holding a catheter and a tube in engagement with tissue.

FIG. 15 is a perspective view of the tissue, catheter and tube of FIG. 13 with the appliance cover, protective cover and integrated backing exploded therefrom.

FIG. 16 is a perspective view of a microstructure feature suitable for use with the appliance covers of FIGS. 10-15.

FIG. 17 is a perspective view of a microstructure array having two microstructures suitable for use with the appliance covers of FIGS. 10-15.

FIG. 18 is a perspective view of a microstructure array having four microstructures suitable for use with the appliance covers of FIGS. 10-15.

FIG. 19 is a perspective view of a wrist band device incorporating microstructures for application of an appliance to skin proximate a wrist.

FIG. 20 is an exploded view of the wrist band device of FIG. 19 showing an appliance attached to a band.

FIG. 21 is a perspective view of an interior of the wrist band device of FIG. 19 showing microstructures.

FIG. 22 is a close-up view of a microstructure of FIG. 21.

FIG. 23 is a perspective view of an appliance device configured to attach a catheter to tissue using a plurality of microstructure arrays attached to a backing.

### DETAILED DESCRIPTION

To secure appliances, a variety of devices are used. These are sometimes referred to as securement or stabilization devices, especially those designed to attach catheters and surgical drains. The devices include adhesives tapes and bandages. They can sometimes detach from the skin causing the appliance to dislodge. This occurs when they get wet or exposed to skin movement. Adhesives, such as Mastisol®, are also sometimes used with tapes and bandages to secure them to the skin. They can be effective, but the product is expensive and is messy to use. Adhesive based products such as tapes, bandages, and Mastisol degrade over relatively short periods of time and thus these products have limited wear time on the skin. They can require frequent removal and application of new tapes, bandages, and adhesives, which can add costs and resources to patient care.

Appliances can also be attached using sutures. For example, many catheters incorporate wings along their sides that allow sutures to be inserted through apertures in the wings to secure catheters. Sutures are effective, but they require skills to use, are painful, can cause skin irritation, and may lead to infections.

As an alternative, devices incorporating microstructures can be used to attach appliances to the skin. Microstructures can be used to penetrate the skin and enter its dermal layers. This provides the ability to secure attachment to the skin. Microstructures include microhooks, microneedles, microstaples, microanchors, and other shapes. The microstructures are typically affixed to a backing, which may contain an adhesive, to which they are attached. Alternatively, the microstructures may be incorporated into an appliance itself. The devices are typically applied in a similar manner to a bandage to the skin.

Microstructures made in the form of microstaples consist of miniature staples and are especially effective in remaining attached to skin for long periods lasting several days to weeks.

Microstructures' superior holding strength make them less prone to detachment than adhesives or tapes. The structures are made out of materials, such as metal or polymers. Consequently, they are not subject to losing their attachment properties in a moist environment, which is a major issue with adhesives and tapes. In contrast to tapes and adhesives, the microstructures typically cause little to no pain upon their application and removal. They also do not cause the skin damage that is observed with tapes and adhesives that can lead to blisters, ulcers, and infections.

The individual microstructures are much smaller in width and diameter than sutures or staples and typically do not penetrate as deeply into the skin. They thus cause less trauma to underlying tissues and its resultant complications, including infection, bleeding, and scar formation. They also cause minimal to no pain upon insertion, while wearing, and when removing the devices. This is because of their miniature size with the tip being the size of a human hair. In addition, they only penetrate to a depth of 1 mm compared to 3-4 mm with conventional stapes and sutures. Since blood vessels and nerve pain fibers are located at 1.5 mm skin depth and beyond, microstructure-based devices do not reach vessels and fibers that cause bleeding and pain, respectively.

Properly shaped microstructures, such as microstructures, attach to the skin in a manner that provide holding strength similar to sutures without the trauma associated with sutures. Application of microstructures is relatively painless, simple and straightforward in contrast to using sutures and staples. Finally, they can be easily affixed directly to an appliance itself or to a device that attaches the appliance to the skin. All of these attributes makes them more attractive than sutures or staples for attaching appliances to skin.

Examples of appliances with openings or a hollow space through which fluid, air, wire or probe pass through are suitable for use with the devices of the present application include tubes, bags, catheters, ostomy devices, drains, diagnostic devices, monitoring devices, drug delivery devices, ventilatory masks, electrodes, and the like.

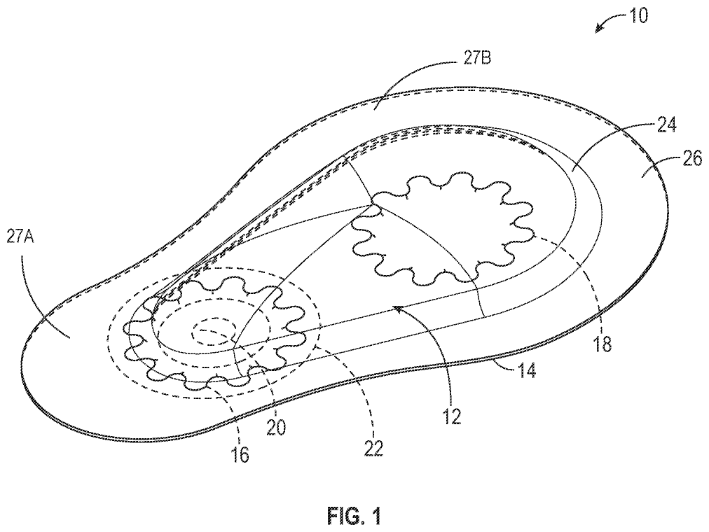

In one example of a skin attachment device, microstaples and other microstructures can be incorporated into an appliance, such as for direct attachment to tissue, such as in the examples of FIGS. 1-4B.

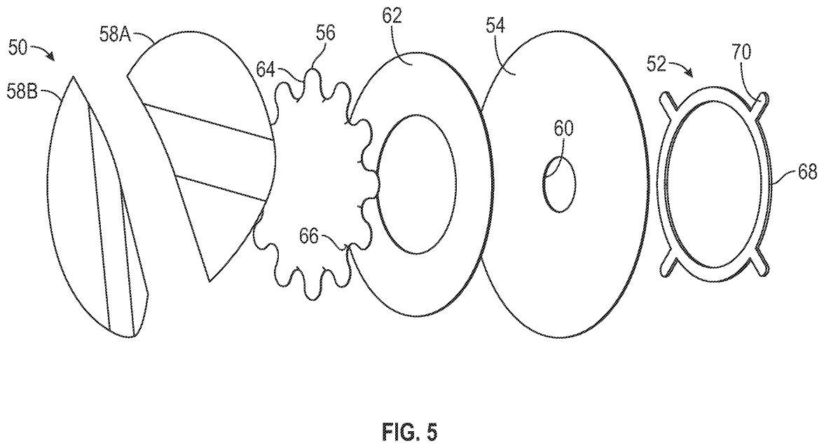

In a second example, a skin attachment device can be attached to the skin itself, such as via microstructures, and then secured to an appliance in a second step, such as for indirect attachment to tissue, such as in the examples of FIGS. 5-9B.

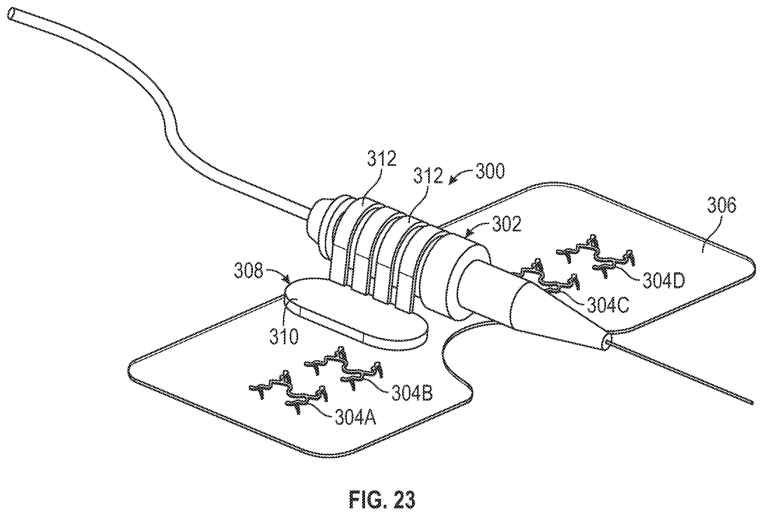

In a third example, the skin attachment device can be affixed to an appliance via a strap, tape or adhesive or staple or suture, and the skin attachment device subsequently attaches to tissue via microstructures, such as in the example of FIG. 23.

In a fourth example, a skin attachment device can be attached to the skin via suture, staple, adhesive or tape.

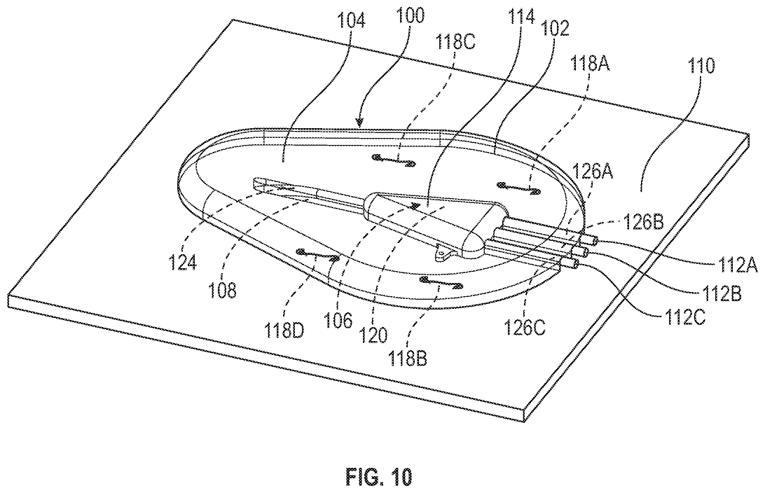

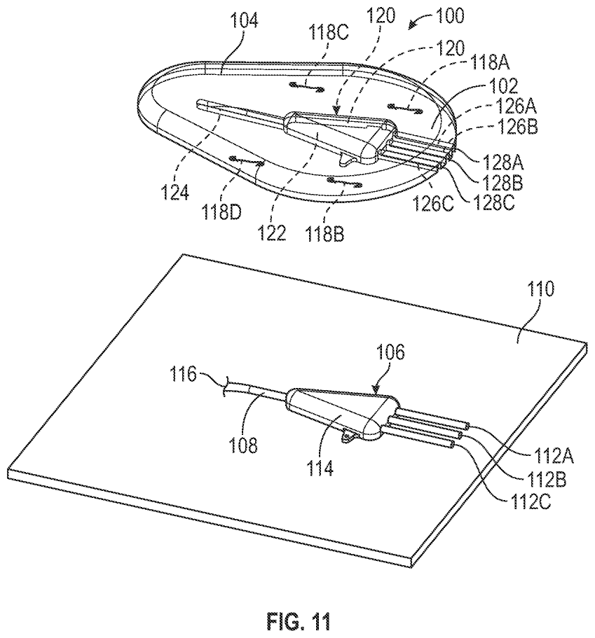

In a fifth example, a skin attachment device is a cover that is used to secure an appliance to the skin without requiring its direct attachment to the appliance. The cover is applied, such as over the top of the appliance after the appliance is in place, and attached to the skin via microstructures, such as in the examples of FIGS. 10-15.

In a sixth example, the cover can be attached to the appliance after the appliance is attached to the skin.

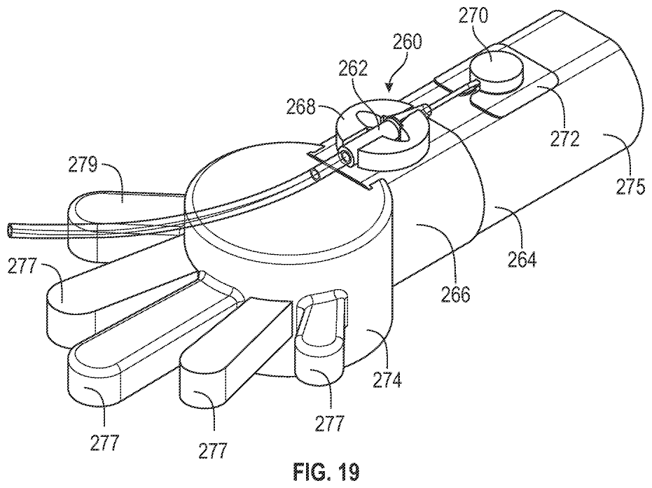

In a seventh example, a skin attachment device can be incorporated into an arm band or wrist band, such as in the examples of FIGS. 19-22.

The microstructures can be of any size, shape, or design that is able to successfully secure the appliance the skin. The microstructures can be any length that allows secure anchoring to the skin. The design of the entire device can contain any number of microstructures that enable secure skin attachment. The design can also contain spacing of the microstructures to allow secure attachment to the skin. The device is designed such that secure skin attachment is achieved without causing significant skin damage or pain. The device may or may not contain adhesive materials or tapes to enhance its utility. It can also be used with or without sutures or staples.

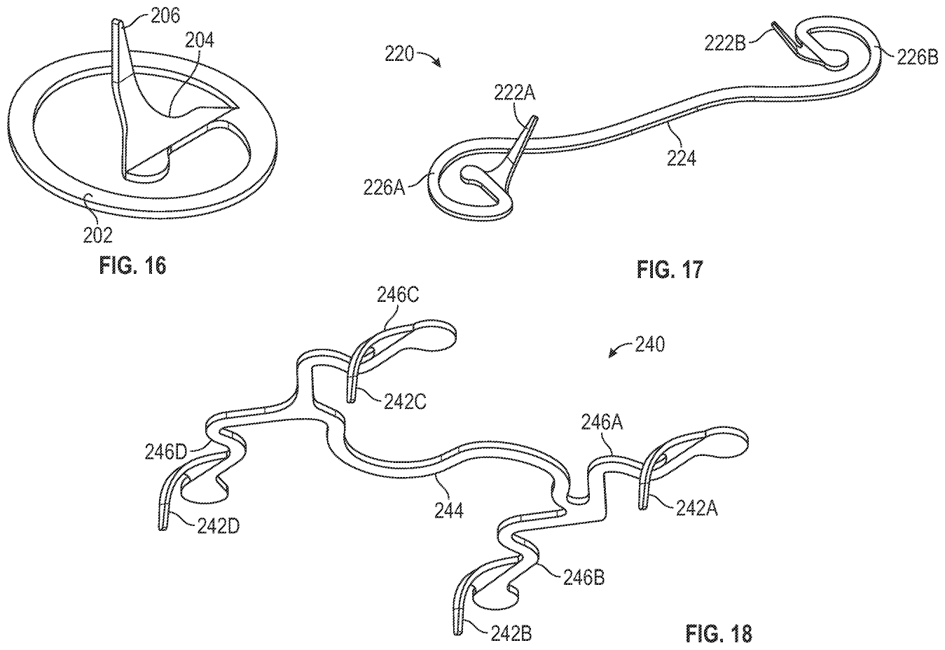

In various examples, microstructures suitable for use with the devices of the present application are described in U.S. patent application Ser. No. 15/446,999 (which published as Pub. No. US 2017/0333039), filed Mar. 1, 2017, International Application No. PCT/US2013/046181, entitled “Microstructure-based wound closure devices,” filed on Jun. 16, 2013, and U.S. Provisional Patent Application No. 62/786,726, filed Dec. 31, 2018, which are hereby incorporated herein by reference in their entirety. Various examples of microstructures suitable for use with the present disclosure are also shown in FIGS. 16-18.

The attached figures show features that can be used in various examples of the microstructure and attachment devices described herein, via illustration of exemplary coupling and appliance devices.

The devices can comprise a base layer, an appliance attached to an air side of the base layer, an adhesive layer attached to a skin side of the base layer and a microstructure attached to the adhesive layer. The appliance can be directly mounted to the base layer, such as shown in FIG. 1. The appliance can be indirectly coupled to the base layer via a coupling features, such as shown in FIG. 5. In examples, the coupling can comprise a flange, a ring, a threaded coupling, hook and loop material, a buckle, and the like.

The attachment devices can also be used to attach appliances to other tissues, such as muscle, tendon, fascia, dermis, subcutaneous fat, intestine, stomach, kidney, heart, lung, liver, brain, pancreas, blood vessels, and others. The attachment device can be attached to these tissues in humans as well as in animals.

### Catheters, Surgical Drains Tubes, and Masks

More specific examples of appliances that are suitable for use with the devices of the present application include catheters, chest tubes, surgical drains, nasogastric tubes, enterostomy tubes, ventilatory masks, and tubing, and other devices that are hollow or contain a hole through which fluid, a probe, wire or air passes through.

Tapes and bandages are often used to secure urinary, peritoneal, hemodialysis and vascular catheters, surgical drains, nasogastric and enterostomy tubing, and chest tubes in place. These devices are all subject to similar risks of dislodgement with adverse clinical consequences. For example, if these devices are dislodged, they provide a portal to entry of bacteria and other microbes that cause infection. Trauma around the site also contributes to infection. Leakage of fluid from drains may be infected which can lead to secondary infections. Dislodgement of urinary catheters can result in bladder distension and increased risk of infection. Dislodgement of chest tubes can not only lead to infection, but also accumulation of fluid compromising respiratory function. Tapes and adhesives are damaging to the skin resulting in blisters and ulcers in some patients. Finally, the need for increased frequency of changing bandages and tapes due to their short wear time increases resource demands, risks of skin trauma, and infections.

Alternatively, sutures are used to secure drains, chest tubes and catheters to the skin. Sutures can cause skin damage, carry risk of infection, and are not only painful upon their application but when they remain on the body. Sutures perform poorly on fragile skin due to the tearing of the skin that occurs when they are tied. Although they can provide attachment of these devices, they are suboptimal for the reasons listed above.

Nasogastric tubes are used for enteral feeding, administering medications, decompressing the stomach and intestine during obstructions, and to drain blood and other fluids from the gastrointestinal tract, The tubes are typically attached to the skin using tapes and bandages which are suboptimal for the same reasons as for attaching other devices. In addition, they can detach from the skin resulting in complications that can be serious in obstructed patients or those with gastrointestinal bleeding. Microstructure based securement devices provide secure and painless skin attachment for these tubes that often remain in for several weeks.

Enterostomy tubes are mostly used as feeding tubes to administer nutrients and vitamins. The tube is inserted through the abdomen into the intestine. It is often attached in place with sutures, which can be associated with pain, risk of infection, and skin irritation. remove materials from the gastrointestinal tract.

There are a variety of respiratory devices that require secure attachment to allow their proper function. Face masks are used with non-invasive ventilation, continuous positive airway pressure (CPAP), and bilevel positive airway pressure (BIPAP). Theses masks are attached to the face using straps that often do not fit tightly resulting in dislodgement of the mask. Endotracheal tubes are used for mechanical ventilation. They are also attached using straps. If too tight, the straps can damage the skin. If too loose, inadequate ventilation occurs which in the worst cases result in dislodgement of the endotracheal tube. Dislodgement of masks and ventilator tubes can have disastrous consequences resulting in hypoxia that can result in brain damage, heart failure, and death in severe cases. Microstructure-based devices provide an attractive alternative to these securement devices. They enable secure attachment of the mask or tube eliminating the risk of hypoxia. Moreover, they can provide consistent and uniform tension eliminating the risk of too loose or too tight attachment to the skin.

### Catheters

Catheters are of primarily three types—vascular, urinary and peritoneal. Vascular catheters include central venous catheters, peripherally inserted central catheters (PICC), and peripheral intravenous catheters, arterial catheters including radial and femoral arterial catheters, and hemodialysis catheters. Secure attachment of vascular catheters to the skin is essential for their proper function and for safety reasons. If catheters detach from the skin, they are at risk of dislodgement from the vessel in which they are inserted. This can result in bleeding, which in severe cases result in significant blood loss requiring transfusions. More seriously, such blood loss can result in hypotension resulting in organ failure and death. In addition, when catheters detach from the skin, this can lead to movement of the catheter where it enters the skin. The movement causes trauma that can increase the risk of infection. It also serves as a nidus for formation of thrombi that can embolize resulting in damage to lungs, limbs, and other parts of the body.

To help address the issues with current devices, we have invented a device that incorporates microstructures to secure appliances to the skin. The microstructures penetrate the skin and provide more secure anchoring to skin than can be achieved with bandages and tapes. They are more resistant to detachment when exposed to moisture or body movement. Microstructure-based devices are less painful when they are placed or worn on the skin,

SecurAcath is a newer securement device that uses prongs that are inserted into the catheter port site to secure the device to the subcutaneous tissue. This provides more secure attachment than other devices, but it is expensive and difficult to use. These devices also require an adhesive backing or cover to secure them in place.

To address the limitations of current devices, a device is required that is easy to use and provides secure skin attachment for extended time periods, while minimizing skin irritation and trauma and risk of infection. Devices incorporating microstructures achieve securement of catheters by penetration into the skin. Devices remain attached to the skin when exposed to moisture resulting from sweat, bleeding, drainage and exposure to water with showering and bathing. Microstructure-based devices thus achieve secure and long-term attachment to skin. In addition, microstructures are much smaller than sutures and thus reduce the risk of trauma and infection. Microstructures attached to an elastic backing are ideal since they allow devices to remain attached during skin movement. This also reduces skin irritation that occurs with most adhesive bandages and tapes. Finally, microstructures can be directly incorporated into appliances or attached to them. Application of the microstructures to secure the device is simple to perform using a method similar to applying a bandage to the skin. This is especially the case when they are attached to a backing.

In a first example, the securement device is directly attached to the appliance.

In a second example, the securement device is a stand-alone device that requires the appliance to be attached to it.

In a third example, the securement device and the appliance are each provided individually in a kit.

In a fourth example, the microstructure-based securement device contains a backing, preferably adhesive-based, which contains microstructures enabling secure skin attachment.

In a fifth example, the catheter securement device consists of two components, including a first component that comprises an adhesive backing containing microstructures that attaches to the skin and a second component that attaches to the appliance and helps secure it in place.

In a sixth example, this second component can be incorporated into the first component to form the securement device.

In a seventh example, the first and second components are provided individually and then subsequently attached to one another.

In the eighth example, the first and second components are provided in a kit.

In a ninth example, the first component can be attached to the skin and then the second component attached to it or the first component can be attached to the second component before it is attached to the skin. The catheter can be attached to the second component before or after attachment to the skin.

In a tenth example, the securement device comprises an adhesive backing containing microstructures that is incorporated into a cover over the catheter.

In an eleventh example, the securement device covers another device that directly attaches to the appliance to secure it.

In an twelfth example, more than one appliance is attached to the securement device.

In a thirteenth example, the cover containing adhesive backing with microstructures is incorporated into the attachment to the catheter and the combination forms a single securement device.

In a fourteenth example, the cover and component that directly attaches to the catheter are each provided individually in a kit.

In a fifteenth example, the microstructure based cover contains a molded piece that conforms to the shape of a catheter.

In a sixteenth example, the microstructure based cover contains a molded piece that conforms to the shape of a component that directly attaches to a catheter.

In a seventeenth example, the molded piece that conforms to the shape of a component is attached to the component that directly attaches to a catheter.

In an eighteenth component, the molded piece that conforms to the shape of a component is not attached to the component that directly attaches to a catheter.

In a nineteenth example, the molded piece is able to conform to different sized catheter attachment devices.

In a twentieth example, the molded piece contains foam material to enable it to conform to different sized catheter attachment devices.

In a twenty first example, the cover and molded component are combined into one catheter securement device.

In a twenty second example, the cover and molded component are provided individually in a kit.

In a twenty third example, the cover, molded piece, and component comprise a catheter securement device.

In a twenty fourth example, the cover, molded piece, and component that directly attaches to the catheter are each provided individually in a kit.

In a twenty fifth example, the micro-structure based securement device includes an arm band that attaches either directly or indirectly to the appliance.

In a twenty sixth example, the arm band contains microstructures.

In a twenty seventh example, more than one appliance is attached to a microstructure-based securement device.

In a twenty eighth example, more than one catheter is attached to a microstructure-based securement device.

In a twenty ninth example, an appliance can include a microstructure-based securement device and sutures, staples, tapes, or bandages to attach it to the skin.

In a thirtieth example, an appliance can include a micro-based securement device and sutures to attach it to skin.

In a thirty first example, an appliance can include a micro-based securement device and staples to attach it to skin.

In a thirty second example, an appliance can include a micro-based securement device and tape to attach it to skin.

In a thirty third example, an appliance can include a micro-based securement device and bandage to attach it to skin.

In a thirty fourth example, the vascular catheter securement device includes a first component that is directly or indirectly attached to the catheter and a second component that covers the port site where the catheter enters the skin.

In a thirty fifth example, the second component is a dressing.

In a thirty sixth example, the cover of the port site contains microstructures.

In a thirty seventh example, the dressing of the port site contains an anti-septic or anti-microbial agent.

In a thirty eighth example, the antiseptic agent is chlorhexidine.

In a thirty ninth example, the first component and the second component comprise one device.

In a fortieth example, the first component and the second component are separate components of the vascular catheter device.

In a forty first example, the first component and the second component are provided individually in a kit.

In a forty second example, the vascular catheter securement device includes a cover over the first and second components.

### Combination of Port Site Closure and Securement Device Catheters

Securement devices are usually affixed at a distance of at least 2 cm from the actual port site where the catheter enters the skin. This distance is required to reduce the risk of infection that could occur from the securement device should it become infected. This space contains catheter area that is not attached to the skin and is at risk of dislodgement from the vasculature with all of its complications. More importantly, the area in which the catheter enters the skin can serve as a nidus of infection. To prevent this from occurring, a second attachment device is often required that covers the port site. In some cases, this second device is attached directly over the port site. An example of such a device is Biopatch®, which is an adhesive-based disc-shaped patch. Alternatively, the entire area including the catheter securement device and the port site are covered with a single cover. An example of such a device is Tegaderm®. In many cases, an antiseptic such as chlorhexidine is contained the device that covers the port site. Often a specific cover is placed over the port site itself and a second cover is applied over the entire skin area that includes the securement site and the port site.

As an alternative, a single microstructure-based securement device can be used to both secure the catheter at a point distal from the port site—the catheter securement device—and also at the port site to provide further security and stabilization of the device as well protecting the catheter from causing infection or serving as a nidus for thrombotic formation that can lead to embolic events.

In a first example, a single microstructure-based device can be used to attach the catheter to the skin 2 cm or more distal to the port site and also attach to the area around the port site to cover the port site.

In a second example, a single microstructure-based securement device can be used to both attach the catheter to the skin 2 cm or more distal to the port site and also attach the catheter at the port site.

In a third example, the device would contain an anti-bacterial or antiseptic agent to reduce the risk of infection.

In a fourth example, the device would contain chlorhexidine.

### Radial Arterial Catheters

Arterial catheters carry a catastrophic risk of bleeding because arterial blood pressure is high. Dislodgement of the catheter results in massive blood loss that can cause hypotension leading to death.

In contrast to venous catheters, this catheter securement device will be typically less than 2 cm from the port site. Although the distance is shorter than with venous catheters, dislodgement of the catheter caused by pulling on the open area between where it is secured on the skin and the port site can still occur. In contrast to venous catheters, there are relatively few securement devices that are used to secure radial arterial catheters. Securement of radial arterial catheters is usually achieved using sutures, tapes, or bandages.

The space between which the catheter enters the skin and the hand is relatively limited and thus devices must be of small size to fit into this area. This is a limitation for most vascular securement devices that require adhesive backing or tapes for skin attachment, because of their limited holding strength. With less adhesive backing attaching the device to the skin, there is a higher risk of detachment.

A microstructure-based device primarily depends on the microstructures, which provides more secure holding strength than adhesive backing to securely attach to the skin. Therefore, reduced adhesive backing surface will have minimal impact on its ability to attach to the skin. Consequently, a small microstructure-based securement device can be used to attach the radial arterial catheter to the skin.

In a first example, a microstructure-based securement device can be used to attach a radial artery catheter.

In a second example, the radial arterial catheter device can contain a wrist or arm band that encircles the distal part of the extremity just proximal to the wrist joint.

In a third example, the wrist band can fully encircle the circumference of the wrist.

In a fourth example, the wrist band can partially encircle the circumference of the wrist.

In a fifth example the component that attaches to the catheter and the wrist band are combined into one securement device.

In a sixth example the component that attaches to the catheter and wrist are separate pieces.

In a seventh example, the component that attaches to the catheter and wrist are provided individually in a kit.

In an eighth example, the wrist band contains microstructures.

In a ninth example, the wrist band can contain adhesive.

In a tenth example, the wrist band contains adhesive and microstructures.

In an eleventh example, the wrist band is adjustable so that it can be fitted to wrists of different circumferences.

In a twelfth example, the wrist band does not contain adhesive.

In a thirteenth example, the microstructures are attached to an elastic backing enabling it to remain in place with movement or edema in the wrist area.

In a fourteenth example, the wrist band does not contain microstructures.

In a fifteenth embodiment, the microstructures are attached to an elastic backing enabling it to be attached so that it can remain in place with movement or edema in the wrist area.

In a sixteenth example, the wrist band does not contain microstructures.

FIG. 1 is a perspective view of appliance device 10 using microstructure attachment features for directly coupling an appliance to tissue. Appliance device 10 can comprise appliance 12, elastic backing 14, microstructure array 16, microstructure array 18, patient interaction feature 20 and adhesive layer 22. Appliance 12 can comprise appliance enclosure portion 24 and base portion 26. Appliance enclosure portion 24 can comprise superior portion 27A and inferior portion 27B.

Appliance 12 can comprise any device configured to interact with a patient, tissue of a patient, biological matter emitted from the patient and the like. In the illustrated example, appliance 12 can comprise an ostomy bag. As such, appliance 12 can comprise a container configured to hold a fluid. For example, appliance enclosure portion 24 can comprise a wall spaced from backing 14 to allow fluid to be retained therebetween. Appliance 12 can be made of a material that is impermeable to fluid. Appliance 12 can comprise a rigid or flexible material. In the illustration of FIG. 1, appliance 12 is illustrated as being transparent. As such, the contents of appliance 12 can be viewed from outside of appliance device 10. In examples, appliance 12 can be opaque or not transparent. For an ostomy bag, patient interaction feature 20 can comprise an access port configured to allow fluid to drain from an incision or port in anatomy. However, in other examples of the present disclosure, patient interaction feature 20 can comprise a sensor, a glucose monitor, an EKG monitor, an EEG monitor, a drug-emitting patch and others.

In the illustrated example, backing 14 has a pear shape, with upper or superior portion 27A and lower or inferior portion 27B. Superior portion 27A can comprise a semi-circular shape and inferior portion 27B can comprise a smaller semi-circular shape, with both portions being connected by arcuate segments. Superior portion 27A can include patient interaction feature 20. Inferior portion 27B can be larger, e.g., encompass a larger surface area, than superior portion 27A. Backing 14 can be configured to be positioned on a patient with superior portion 27A and patient interaction feature 20 pointed upward or in the superior direction. As such, fluid collected from the patient through patient interaction feature 20 can drain into inferior portion 27B for collection into appliance enclosure portion 24 of appliance 12. Appliance enclosure portion 24 can also have a pear shape that is smaller than backing 14 or another shape, e.g., two circular arcs connected by two tangent lines, wherein a smaller volume can be positioned superior of a larger volume to direct fluid into a storage volume.

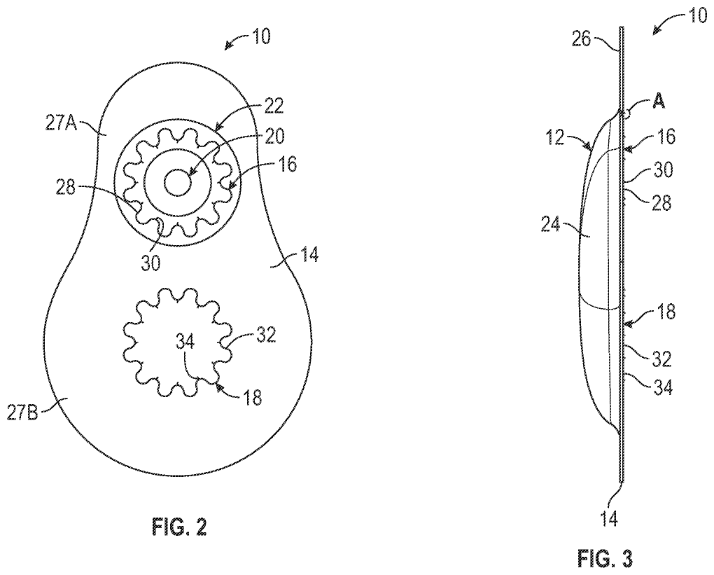

FIG. 2 is a bottom view of appliance device 10 of FIG. 1 showing backing 14, first microstructure array 16, second microstructure array 18, patient interaction feature 20 and adhesive layer 22. Microstructure array 16 can comprise foundation 28 and microstructure 30. Microstructure array 18 can comprise foundation 32 and microstructures 34.

Backing 14 can comprise a stretchable/elastic substrate or base upon which other components of appliance device 10 can be affixed. Backing 14 can comprise a sheet configured to stretch when subject to a tensile load but that will return to its original shape (or close to it) when loading is removed. In an example, backing 14 can be elastic, e.g., a substance or object able to resume its normal shape spontaneously after contraction, dilatation, or distortion.) During use applied to skin, backing 14 may be unstretched, partially stretched or completely stretched.

Backing 14 can be any material such as a fabric or polymer. In some examples, backing 14 can comprise a material singularly, or in combination, selected from the group consisting of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, expanded PTFE (ePTFE), plastic and plastic components, polymers, biopolymers, and natural materials. In examples, backing 14 can comprise a silicone sheet. In examples, devices of the present disclosure can include bases, backings or substrates made in similar to those structures disclosed in US Patent Publication Nos. 2015/0305739 and 2017/0333039. Backing 14 can comprise a waterproof or impermeable material to prevent fluid collected in appliance enclosure portion 24 from leaking out of appliance device 10.

Backing 14 can comprise a continuous material that can be positioned adjacent tissue, except for patient interaction feature 20. Backing 14 can have any shape suitable for attaching a given appliance attached to device 10. Thus, backing 14 can generally conform to the shape of appliance 12. However, backing 14 can have any shape including, but not limited to, rectangular, rectilinear, square, circular, oval, oblong and the like. In the illustrated example, backing 14 and base portion 26 can have the same outer perimeter shape.

Adhesive layer 22 or an adhesive backing can be disposed on the tissue-facing side to allow appliance 10 to be secured to tissue in conjunction with microstructure arrays 16 and 18. The adhesive can be applied uniformly across the entire backing or only around the perimeter of the backing (e.g., backing 14) to ensure good adhesion and sealing to the wound area or interspersed throughout the backing. For example, adhesive layer 22 can comprise only a ring centered to surround patient interaction feature 20. Additionally, a ring of adhesive material can be placed about microstructure array 18. Other adhesive patterns can also be used in this application. The terms adhesive, adhesive layer and adhesive backing are used interchangeably. Microstructure arrays 16 and 18 can be applied to the tissue-facing side against the adhesive layer. The adhesive can include any medical grade adhesive, such as, for example, an acrylate hydrocolloid or silicone.

FIG. 3 is a side view of appliance device 10 of FIG. 2 showing first and second microstructure arrays 16 and 18 attached to a skin-facing side of backing 14 and appliance 12 attached to an air-facing side of backing 14. Microstructure array 16 can comprise foundation 28 and microstructure 30. Microstructure array 18 can comprise foundation 32 and microstructures 34.

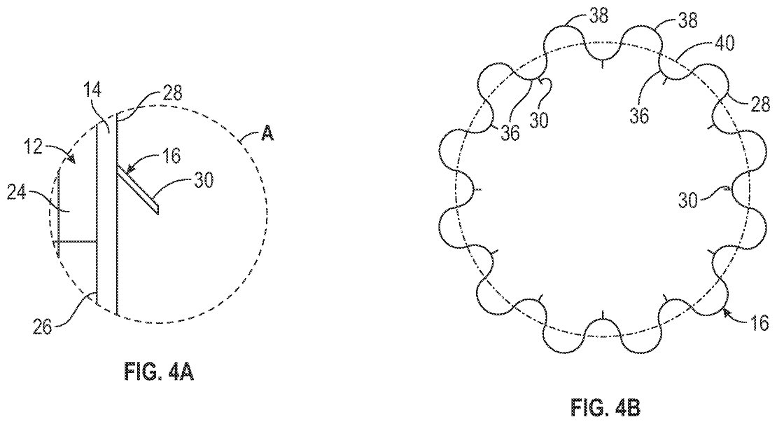

As can be seen in FIG. 3, base portion 26 of appliance 12 can comprise a thin layer stacked against an air side of backing 14. Appliance enclosure portion 24 can protrude from base portion 26 to provide a volume for fluid storage. Base portion 26 can be attached to backing 14 via any suitable method, such as adhesive, glue, chemical bonding, melting and the like. Foundation 28 of microstructure array 16 and foundation 32 of microstructure array 18 can be attached to a skin side of backing 14, such as via an adhesive. Such a configuration can be seen in callout A of FIG. 4A.

FIG. 4A is a closeup view of microstructure array 16 attached to the skin-facing side of backing 14. Microstructure array 16 can comprise foundation 28 and microstructure 30 protruding from a microstructure array attached to the skin-facing side of the backing. Microstructure array 18 (FIG. 2) can similarly comprise foundation 32 and microstructures 34. In the illustrated example, microstructure 30 can be at an angle of approximately 45 degrees relative to backing 14. In examples, microstructures 34 can be perpendicular to backing 14 or less than a 90-degree angle with respect to backing 14.

FIG. 4B is an isolated view of microstructure array 16 showing foundation 28 and microstructures 30. In the illustrated example, foundation 28 can comprise a ring having an undulating shape. For example, foundation 28 can have a plurality of alternating inward facing bumps 36 and outward facing bumps 38 extending from circular centerline 40 spaced equidistant from the outer most tips of the bumps and the inner most tips of the bumps. The undulations formed by the inward and outward facing bumps can provide microstructure array 16 with a spring characteristic that allows microstructure array 16 to stretch and bend. For example, the diameter of microstructure array 16 can contract and expand. The circular shape of foundation 32 can allow microstructure array 16 to circumscribe patient interaction feature 20 (FIG. 2). Thus, microstructure array 16 can be used to hold patient interaction feature 20 (FIG. 2) in close contact with tissue around the entire perimeter of patient interaction feature 20 (FIG. 2). In examples, microstructure array 16 can be replaced with a plurality of foundations, each having a circular arc shaped foundation and a plurality of microstructures. Such foundations could be placed around patient interaction feature 20 (FIG. 2) to provide connection to tissue while still permitting flexibility.

A plurality of microstructures 30 can be provided on foundation 28. A plurality of microstructures 30 can define an array. Microstructures 30 can have a pointed tip. Microstructures 30 can comprise any micro-sized structure suitable for grabbing onto or piercing into tissue, e.g., skin, such as barbs, hooks, anchors, needles, blades, fishscales, pillars, hairs (i.e., a microstaple, a microbarb, a microneedle, a microblade, a microanchor, a microhook, a microfishscale, a micropillar, and a microhair) and the like.

Microstructure array 18 can be configured similarly to microstructure array 16. Microstructure array 18 can be spaced away from microstructure array 16 on backing 14 (FIG. 2) to provide attachment for a different portion of appliance device 10 than microstructure array 16. For example, microstructure array 16 can be positioned substantially within superior portion 27A (FIG. 2) of backing 14 and microstructure array 18 can be positioned substantially within inferior portion 27B (FIG. 2) of backing 14.

Microstructure array 18 and microstructure array 16 are illustrated as having the same configuration, e.g., circular arrays with spring characteristics. However, in examples, microstructure array 16 and microstructure array 18 can have different configurations. In examples, both of microstructure arrays 16 and 18 can both have spring characteristics to provide stretchability of the array to facilitate conformability to anatomy. In an example, microstructure array 18 can be replaced by one or more microstructure arrays described in US Patent Publication Nos. 2015/0305739 and 2017/0333039.

Microstructure arrays 16 and 18 allow backing 14 to be attached to tissue to maintain patient interaction feature 20 in close contact with the tissue to allow effective fluid transfer therebetween without leakage, while also maintaining appliance 12 is a stable and immobilized fashion to inhibit shaking and potential dislodgement.

FIG. 5 is an exploded view of appliance device 50 using microstructure attachment features for indirectly coupling an appliance to tissue via coupler 52. Appliance device 50 can comprise coupler 52, elastic backing 54, microstructure array 56, disposable packaging portions 58A and 58B, patient interaction feature 60 and adhesive layer 62. Microstructure array 56 can comprise foundation 64 and microstructures 66. Coupler 52 can comprise ring 68 and spokes 70, as an example of a locking mechanism or mating feature.

Appliance device 50 can comprise a device configured to allow another device to be held against tissue such as skin or an incision or port within such tissue or skin. In examples, patient interaction feature 60 can comprise an opening in backing 54 to allow passage of material through backing 54. Thus, a substance or component of an appliance can be allowed to pass into tissue from an appliance, or a fluid or matter from the tissue can be allowed to pass into an appliance. In examples, patient interaction feature 60 can comprise a device configured to be held in contact or near contact with tissue, such as one or more of a sensor, microchip, an electronic communication device, a Bluetooth device, an EEG electrode and EKG monitor, an EEG monitor, a mechanical device and the like, and coupler 52 can be configured to hold another component in engagement with patient interaction feature 60.

Coupler 52 can be configured to couple to specific mating features on another device or coupler 52 can be configured to have one or more generic mating features configured to mate with features a plurality of other another devices. Coupler 52 can comprise a coupling, an attachment feature, an appliance securing feature, an appliance structure, a coupling device as is used herein.

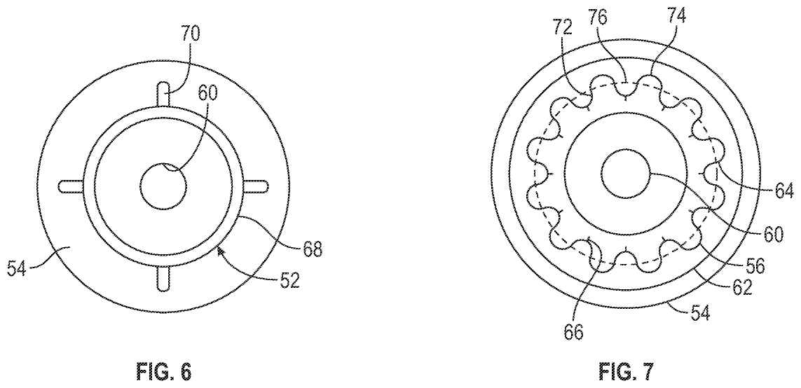

FIG. 6 is a top view of appliance device 60 of FIG. 5 showing patient interaction feature 60 and coupler 52 attached to backing 54. Coupler 52 can comprise ring 68 and spokes 70. Patient interaction feature 60 can comprise a port or hole on backing 54. Patient interaction feature 60 can be centered on backing 54.

Backing 54 can comprise a stretchable/elastic substrate or base upon which other components of appliance device 50 can be affixed. Backing 54 can comprise a sheet configured to stretch when subject to a tensile load but that will return to its original shape (or close to it) when loading is removed. In an example, backing 54 can be elastic, e.g., a substance or object able to resume its normal shape spontaneously after contraction, dilatation, or distortion.) During use applied to skin, backing 54 may be unstretched, partially stretched or completely stretched.

Backing 54 can be any material such as a fabric or polymer. In some examples, backing 54 can comprise a material singularly, or in combination, selected from the group consisting of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, expanded PTFE (ePTFE), plastic and plastic components, polymers, biopolymers, and natural materials. In examples, backing 54 can comprise a silicone sheet. In examples, devices of the present disclosure can include bases, backings or substrates made in similar to those structures disclosed in US Patent Publication Nos. 2015/0305739 and 2017/0333039. Backing 54 can comprise a waterproof or impermeable material to prevent fluid from leaking through of appliance device 50.

Backing 54 can comprise a continuous material that can be positioned adjacent tissue, except for patient interaction feature 60. Backing 54 can have any shape suitable for attaching a given appliance attached to device 50. Thus, backing 54 can generally conform to the shape of coupler 52. However, backing 54 can have any shape including, but not limited to, rectangular, rectilinear, square, circular, oval, oblong and the like. In the illustrated example, backing 54 and coupler 52 can generally have ring shapes.

Adhesive layer 62 or an adhesive backing can be disposed on the tissue-facing side to allow appliance 50 to be secured to tissue in conjunction with microstructure array 56. The adhesive can be applied uniformly across the entire backing or only around the perimeter of the backing (e.g., backing 54) to ensure good adhesion and sealing to the wound area or interspersed throughout the backing. For example, adhesive layer 62 can comprise only a ring centered to surround patient interaction feature 60. Other adhesive patterns can also be used and in this application. The terms adhesive, adhesive layer and adhesive backing are used interchangeably. Microstructure array 56 can be applied to the tissue-facing side against the adhesive layer. The adhesive can include any medical grade adhesive, such as, for example, an acrylate hydrocolloid or silicone.

In examples, patient interaction feature 60 comprises a circular hole centered on backing 54, and backing 54 can have a circular outer perimeter centered on patient interaction feature 60. Ring 68 of coupler 52 can surround patient interaction feature to distribute loading from an appliance attached to appliance device 60 to backing 54 evenly. In examples, ring 68 can be centered around patient interaction feature 60. In examples, ring 68 can be completely attached to backing 54. However, ring 68 can be intermittently attached to backing 54. In examples, ring 68 can be attached to backing 54 via glue, adhesive or other chemical bonding means. In other examples, ring 86 can be mechanically attached to backing 54, such as via rivets or threaded fasteners.

Ring 68 can include other features to facilitate attachment of an appliance to ring 68. In the illustrated example, ring 68 comprises spokes 70. Spokes 70 can comprise lugs or features onto which other components can latch onto. Thus, spokes 70 can be un-directly attached to backing 54 (e.g., such that something can be slipped between spokes 70 and backing 54) so that spokes 70 can be plugged into sockets of an appliance. In examples, the appliance can be twisted after receiving spokes 70 to provide a twist-lock coupling. In other examples, features of the appliance can receive and attach to spokes 70 without slipping behind spokes 70. In additional examples, the inner diameter of ring 68 can be threaded to receive mating threads of an appliance or appliances can be directly threaded onto the inner diameter of ring 68 regardless of threading. Likewise, in additional examples, spokes 70 can be omitted and the outer diameter of ring 68 can provides a land or edge upon which mating components of an appliance can be threaded.

FIG. 7 is a bottom view of appliance device 50 of FIG. 6 showing patient interaction feature 60, microstructure array 56 and adhesive layer 62 attached to backing 54. Microstructure array 56 can comprise foundation 64 and microstructures 66.

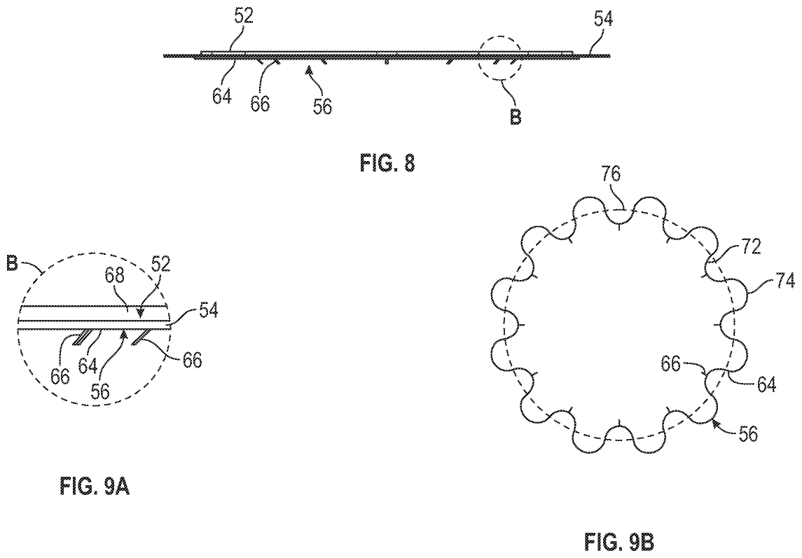

FIG. 8 is a side view of appliance device 50 of FIG. 7 showing microstructure array 56 attached to a skin-facing side of backing 54 and coupler 52 attached to an air-facing side of backing 54.

As can be seen in FIG. 8, coupler 52 can comprise a thin layer stacked against an air side of backing 54. Foundation 64 of microstructure array 56 can be attached to a skin side of backing 54. Such configuration can be seen in callout B of FIG. 9A.

FIG. 9A is a closeup view of foundation 64 attached to backing 54 and microstructure 66 protruding from foundation 64.

In the illustrated example, microstructure 66 can be at an angle of approximately 45 degrees relative to backing 54. In examples, microstructures 66 can be perpendicular to backing 54 or less than a 90-degree angle with respect to backing 54.

FIG. 9B is an isolated view of microstructure array 56 showing foundation 64 and microstructures 66. In the illustrated example, foundation 64 can comprise a ring having an undulating shape. For example, foundation 64 can have a plurality of alternating inward facing bumps 72 and outward facing bumps 74 extending from circular centerline 76 spaced equidistant from the outer most tips of the bumps and the inner most tips of the bumps. The undulations formed by the inward and outward facing bumps can provide microstructure array 56 with a spring characteristic that allows microstructure array 56 to stretch and bend. For example, the diameter of microstructure array 56 can contract and expand. The circular shape of foundation 64 can allow microstructure array 56 to circumscribe patient interaction feature 60. Thus, microstructure array 56 can be used to hold patient interaction feature 60 in close contact with tissue around the entire perimeter of patient interaction feature 60. In examples, microstructure array 56 can be replaced with a plurality of foundations, each having a circular arc shaped foundation and a plurality of microstructures. Such foundations could be placed around patient interaction feature 60 to provide connection to tissue while still permitting flexibility.

A plurality of microstructures 66 can be provided on foundation 64. A plurality of microstructures 66 can define an array. Microstructures 66 can have a pointed tip. Microstructures 66 can comprise any micro-sized structure suitable for grabbing onto or piercing into tissue, e.g., skin, such as barbs, hooks, anchors, needles, blades, fishscales, pillars, hairs (i.e., a microstaple, a microbarb, a microneedle, a microblade, a microanchor, a microhook, a microfishscale, a micropillar, and a microhair) and the like.

In examples, microstructure array 56 can have spring characteristics to provide stretchability of the array to facilitate conformability to anatomy. In an example, microstructure array 56 can be replaced by one or more microstructure arrays described in US Patent Publication Nos. 2015/0305739 and 2017/0333039.

Microstructure array 56 allows backing 54 to be attached to tissue to maintain patient interaction feature 60 in close contact with the tissue to allow effective contact therewith, while also maintaining coupler 52 (FIG. 6) is a stable and immobilized fashion to inhibit shaking and potential dislodgement. Coupler 52 (FIG. 6) can thereafter (or beforehand) allow one or more appliances, e.g., ostomy bags, catheters, monitors, electrodes, sensors, etc., to be held in contact or close to the tissue.

In examples, appliance device 60 can be provided in a kit with an appliance or a plurality of appliances that are each configured to attach to appliance device 60.

FIG. 10 is a perspective view of appliance cover 100 having appliance portion 102 integrated with protective cover 104 configured to hold catheter 106 and tube 108 in engagement with tissue 110. FIG. 11 is a perspective view of tissue 110, catheter 106 and tube 108 of FIG. 10 with appliance cover 100 exploded therefrom. FIGS. 10 and 11 are discussed concurrently.

Tissue 110 can comprise an area of skin, such as a portion of skin over an artery, vein or organ for which catheter 106 is to be used. Catheter 106 can be applied to the exterior of tissue 110. Catheter 106 can comprise a 3-way catheter in which a plurality of inlet and outlet tubes 112A, 112B and 112C are connected to one or more passages within catheter tube 108 via housing 114. In examples, catheter 106 can comprise a urinary catheter, a peritoneal dialysis catheter, a vascular catheter, an arterial catheter, a hemodialysis catheter, a venous catheter, a central venous catheter, a peripherally inserted central catheter (PICC) and a peripheral intravenous catheter. As such, fluid can be exchanged between the interior and the exterior of tissue 110. Catheter tube 108 can be inserted into incision 116 in tissue 110. In order to prevent irritation at incision 116 and ingress and/or egress of fluid, bacteria and the like, it is desirable for housing 114 to maintain stationary relative to tissue 110. Appliance cover 100 can be installed over catheter 106 to immobilize both housing 114 and tube 108 relative to tissue 110. Appliance cover 100 can include microstructure arrays 118A-118D configured to hold appliance portion 102 and protective cover 104 against tissue 110. Microstructure arrays 118A-118D can comprise a plurality of microstructures configured to hold cover 100 into engagement with tissue 110. Microstructure arrays 118A-118D are illustrated as each comprising two microstructures, such as those shown in FIG. 17. However, other types of microstructures can be used, such as those shown in FIGS. 16 and 18 or those described in U.S. patent application Ser. No. 15/446,999 (which published as Pub. No. US 2017/0333039), filed Mar. 1, 2017, and U.S. Provisional Patent Application No. 62/786,726, filed Dec. 31, 2018, which are hereby incorporated herein by reference in their entirety.

Cover 100 can comprise one or more pieces of material configured to extend over housing 114, catheter tube 108 and fluid tubes 112A-112C. In examples, cover 10 can be fabricated from an elastic or compliant material, such as a foam or rubber. In examples, cover 100 can be fabricated from a material that holds or retains its shape and an interior compliant or foam layer can be attached thereto. As such, cover 100 can adapt to the contours of catheter 106. In examples, cover 100 can be particularly adapted to mate with specific appliances. In the illustrated example, cover 100 is configured to operate with catheter 106, with catheter 106 comprising a 3-way bladder catheter. Cover 100 can comprise appliance securing feature 120 configured to receive housing 114, tube 108 and tubes 112A-112C. Appliance securing feature 120 can comprise housing socket 122, catheter tube socket 124 and fluid tube sockets 126A-126C. In examples, cover 100 can comprise a molded component fabricated from rubber, a polymer or the like, wherein appliance securing feature 120 is shaped to closely conform with housing 114, tube 108 and tubes 112A-112C. Socket 122 can be configured to hold housing 114 in a secure fashion, e.g., via an interference fit, or in a loose fashion to permit easy in and out of housing 114. Socket 124 can be configured to receive tube 108 from housing 114 and guide tube 108 toward incision 116. Thus, socket 124 can include a sloping sidewall opposing incision 116 to influence the axial direction of tube 108 toward incision 116. Sockets 126A-126C can be configured to receive tubes 112A-112C from housing 114 and guide tubes 112A-112C out of cover 100. Thus, cover 100 can include exit ports 128A-128C for sockets 126A-126D.

Cover 100 can thus simultaneously hold catheter housing 114 in a fixed relationship relative to tissue 110 and tube 108 in a fixed relationship to incision 116. Microstructure arrays 118A-118D can be used to hold cover 100 in place while experiencing the benefits of microstructures as described herein without some or all of the drawbacks of tape as described herein.

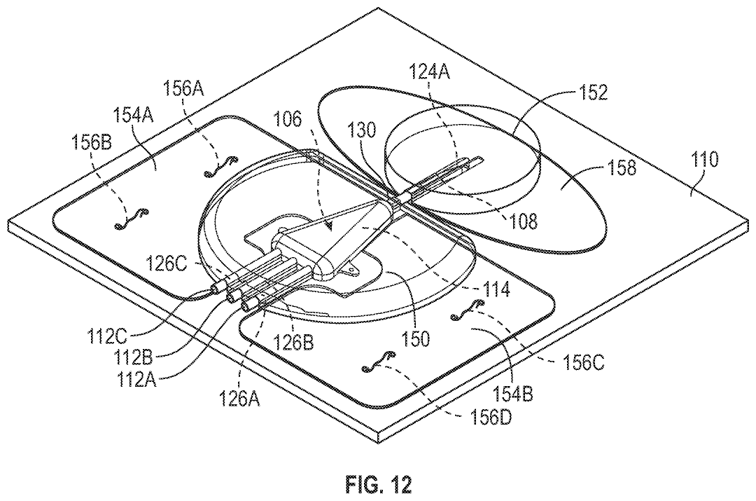

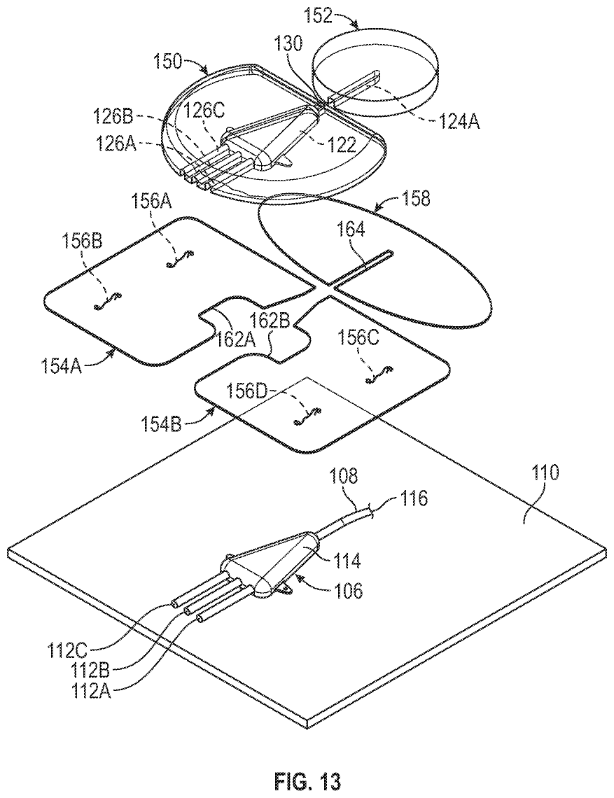

FIG. 12 is a perspective view of appliance cover 150 and separate protective cover 152 for holding catheter 106 and a tube 108 in engagement with tissue 110. FIG. 13 is a perspective view of tissue 110, catheter 106 and tube 108 of FIG. 12 with appliance cover 150 and protective cover 152 exploded therefrom. FIGS. 12 and 13 are discussed concurrently in subsequent paragraphs.

Catheter 106, tube 108 and tubes 112A-112C can be configured similarly as discussed with reference to FIGS. 10 and 11.

Cover 150 can include sockets 122 and 126A-126C as described with reference to FIGS. 10 and 11. However, cover 150 can comprise socket 124A for tube 108. Socket 124A can be truncated to eliminate the sloping sidewall of socket 124. Instead, socket 124A can include exit port 130 to allow tube 108 to extend out of cover 150 and into protective cover 152. As with cover 100, cover 150 can comprise a molded component configured to mate with a particular appliance, e.g., catheter 106. However, in other examples, similar to cover 100, cover 150 can include a pliable layer, e.g., foam, that can conform to other shaped appliances.

Protective cover 152 can comprise a component separate from cover 150. Protective cover 152 can comprise inlet 160 to allow tube 108 to enter socket 162. Socket 162 can be configured similarly as socket 124 of cover 110. Thus, socket 162 can comprise a sloping sidewall configured to guide tube 108 into incision 116.

Protective cover 152 can be fabricated of an absorbent material to absorb fluid emitting from incision 116. In examples, protective cover 152 can be fabricated from cotton or gauze. Protective cover 152 can be configured to be changed out as needed or desired to keep incision 116 clean. Protective cover 152 can be infused with materials or liquids to help keep incision 116 antiseptic, such as chlorhexidine. Protective cover 152 can be configured as a disposable component. Cover 150 can be provided in a kit with a plurality of protective covers 152.

Cover 150 can comprise backing 154A and backing 154B, microstructure arrays 156A-156D. Protective cover 152 can comprise backing 158. Backings 154A and 154B can comprise a sheet of material configured to attach cover 150 to microstructure arrays 156A-156D. Cover 150 can be attached to backings 154A and 154B via any suitable method, such as adhesives, glue, chemical bonding and the like, or via mechanical means such as rivets or fasteners. The tissue-facing side of backings 154A and 154B can comprise an adhesive layer or coating configured to facilitate attachment of microstructure arrays 156A-156D to backings 154A and 154B, as well as to facilitate attachment of backings 154A and 154B to tissue 110. Backings 154A and 154B can include cut-outs 162A and 162B configured allow backings 154A and 154B to attach to cover 150 while allowing housing 114 of catheter to engage tissue.

Backing 158 can comprise a sheet of material configured to attach protective cover 152 to tissue 110. Protective cover 152 can be attached to backing 158 via any suitable method, such as adhesives, glue, chemical bonding and the like, or via mechanical means such as rivets or fasteners. The tissue-facing side of backing 158 can comprise an adhesive layer or coating configured to facilitate attachment of backing 158 to tissue 110. Backing 158 can include cut-out configured to allow tube 108 to reach incision 116.

Backings 154A, 154B and 158 can comprise stretchable/elastic substrates or bases upon which other components of appliance cover 150 and protective cover 152 can be affixed. Backings 154A, 154B and 158 can comprise sheets configured to stretch when subject to a tensile load but that will return to its original shape (or close to it) when loading is removed. In an example, backings 154A, 154B and 158 can be elastic, e.g., a substance or object able to resume its normal shape spontaneously after contraction, dilatation, or distortion.) During use applied to skin, backings 154A, 154B and 158 may be unstretched, partially stretched or completely stretched.

Backings 154A, 154B and 158 can be any material such as a fabric or polymer. In some examples, backings 154A, 154B and 158 can comprise a material singularly, or in combination, selected from the group consisting of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, expanded PTFE (ePTFE), plastic and plastic components, polymers, biopolymers, and natural materials. In examples, backings 154A, 154B and 158 can comprise a silicone sheet. In examples, devices of the present disclosure can include bases, backings or substrates made in similar to those structures disclosed in US Patent Publication Nos. 2015/0305739 and 2017/0333039. Backing 154A, 154B and 158 can comprise a waterproof or impermeable material to prevent fluid from passing therethrough.

Microstructure arrays 156A-156D are illustrated as each comprising two microstructures, such as those shown in FIG. 17. However, other types of microstructures can be used, such as those shown in FIGS. 16 and 18 or those described in U.S. patent application Ser. No. 15/446,999 (which published as Pub. No. US 2017/0333039), filed Mar. 1, 2017, and U.S. Provisional Patent Application No. 62/786,726, filed Dec. 31, 2018, which are hereby incorporated herein by reference in their entirety.

Cover 150 can thus hold catheter housing 114 in a fixed relationship relative to tissue 110 while protective cover 152 holds tube 108 in a fixed relationship to incision 116. Microstructure arrays 156A-156D can be used to hold cover 150 in place while experiencing the benefits of microstructures as described herein without some or all of the drawbacks of tape as described herein. However, protective cover 152 can be easily applied and removed from tissue 116 via an adhesive layer on backing 158.

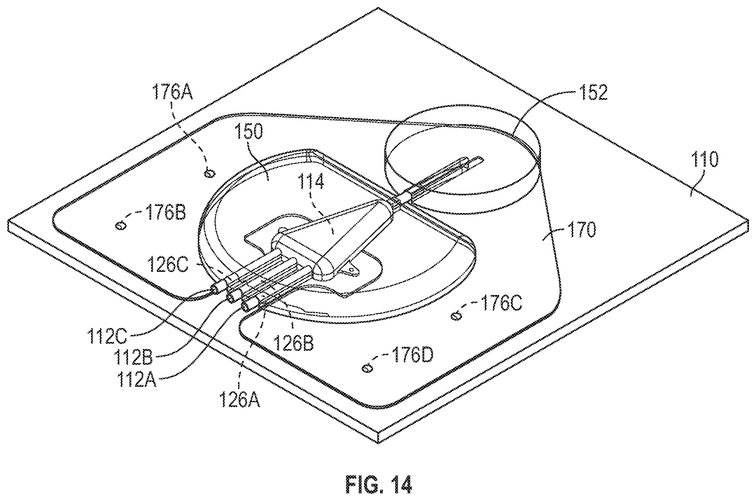

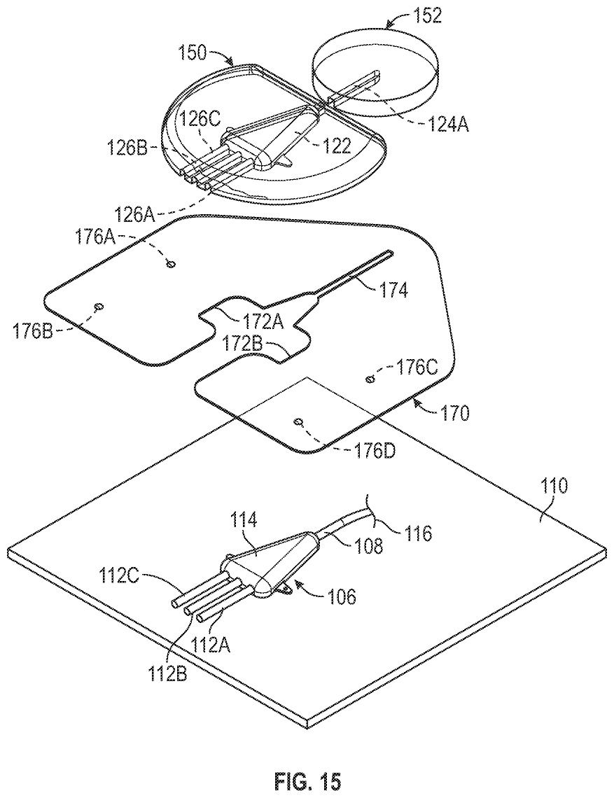

FIG. 14 is a perspective view of appliance cover 150 and separate protective cover 152 with integrated backing 170 for holding catheter 106 and tube 108 in engagement with tissue 110. FIG. 15 is a perspective view of tissue 110, catheter 106 and tube 108 of FIG. 13 with appliance cover 150, protective cover 152 and integrated backing 170 exploded therefrom. FIGS. 13 and 14 are discussed concurrently.

Catheter 106, including tube 108, tubes 112A-112C and housing 114, can be configured similarly as described with reference to FIGS. 10 and 11. Appliance cover 150 and protective cover 152 can be configured similarly as described with reference to FIGS. 12 and 13.

Integrated backing 170 can replace backings 154A, 154B and 158 of FIGS. 12 and 13. Integrated backing 170 can include cut-outs 172A and 172B that can be similarly shaped to cut-outs 162A and 162B. Integrated backing 170 can also include cut-out 174 that can be similarly shaped to cut 164. Protective cover 152 can be attached to integrated backing 170 via adhesive.

Integrated backing 170 can comprise microstructures 176A-176D. Microstructures 176A-176D can comprise a plurality of microstructures configured to hold integrated backing 170 into engagement with tissue 110. Microstructures 176A-176D are illustrated as each comprising a single microstructure, such as those shown in FIG. 16. However, other types of microstructures can be used, such as those shown in FIGS. 17 and 18 or those described in U.S. patent application Ser. No. 15/446,999 (which published as Pub. No. US 2017/0333039), filed Mar. 1, 2017, and U.S. Provisional Patent Application No. 62/786,726, filed Dec. 31, 2018, which are hereby incorporated herein by reference in their entirety.

Integrated backing 170 can thus allow cover 150 to be tightly secured to tissue 110 and provide a base upon which protective cover 152 can be mounted. Backing 170 can thus provide protection to tissue 110 from repeated applications and removal of multiple instances of protective cover 152 as each becomes soiled and needs to be replaced.

FIG. 16 is a perspective view of microstructure feature 200 suitable for use with the appliance covers of FIGS. 10-15. Microstructure feature 200 can comprise foundation 202, arm 204 and prong 206. Foundation 202 can comprise structure with adequate surface area to facilitate attachment of microstructure feature 200 to a backing, a cover or another object. Thus, foundation 202 provides surface area for adhesive to attach. Foundation 202 can comprise a ring-shaped body that surrounds prong 206. The ring-shaped body can allow for the distribution of loads between prong 206 and the component to which microstructure feature 200 is attached. Arm 204 can extend from foundation 202 to position prong 206 within foundation 202, such as proximate the center of the ring-shaped body. Prong 206 can comprise a microstructure as described herein that has a sharpened point to penetrate tissue. Prong 206 can comprise any micro-sized structure suitable for grabbing onto or piercing into tissue, e.g., skin, such as barbs, hooks, anchors, needles, blades, fishscales, pillars, hairs (i.e., a microstaple, a microbarb, a microneedle, a microblade, a microanchor, a microhook, a microfishscale, a micropillar, and a microhair) and the like.

Prong 206 can be straight or curved. Prong 206 can be angled such that the tip of prong 206 can be at an angle of approximately 45 degrees relative to foundation 202. In examples, prong 206 can be perpendicular to foundation 202 or less than a 90-degree angle with respect to foundation 202. Microstructure feature 200 can be fabricated from a single piece of material or as a monolithic material.

FIG. 17 is a perspective view of microstructure array 220 having microstructures 222A and 222B suitable for use with the appliance covers of FIGS. 10-15. Micro-structure array 220 can comprise microstructures 222A, 222B, bridge 224, spring structure 226A and spring structure 226B. A plurality of micro-structures can define an array. Microstructures 222A and 222B can comprise any micro-sized structure suitable for grabbing onto or piercing into tissue, e.g., skin, such as barbs, hooks, anchors, needles, blades, fishscales, pillars, hairs (i.e., a microstaple, a microbarb, a microneedle, a microblade, a microanchor, a microhook, a microfishscale, a micropillar, and a microhair) and the like. Spring structures 226A and 226B can allow stretchability and conformability to microstructure array 220.

FIG. 18 is a perspective view of a microstructure array 240 having four microstructures 242A-242D suitable for use with the appliance covers of FIGS. 10-15. Micro-structure array 240 can comprise microstructures 242A-242D, spring structure 244 and spring structure 246A-246D. A plurality of microstructures 242 can define an array. Microstructures 222A and 222B can comprise any micro-sized structure suitable for grabbing onto or piercing into tissue, e.g., skin, such as barbs, hooks, anchors, needles, blades, fishscales, pillars, hairs (i.e., a microstaple, a microbarb, a microneedle, a microblade, a microanchor, a microhook, a microfishscale, a micropillar, and a microhair) and the like. Spring structures 246A-246D can allow stretchability and conformability to microstructure array 240.

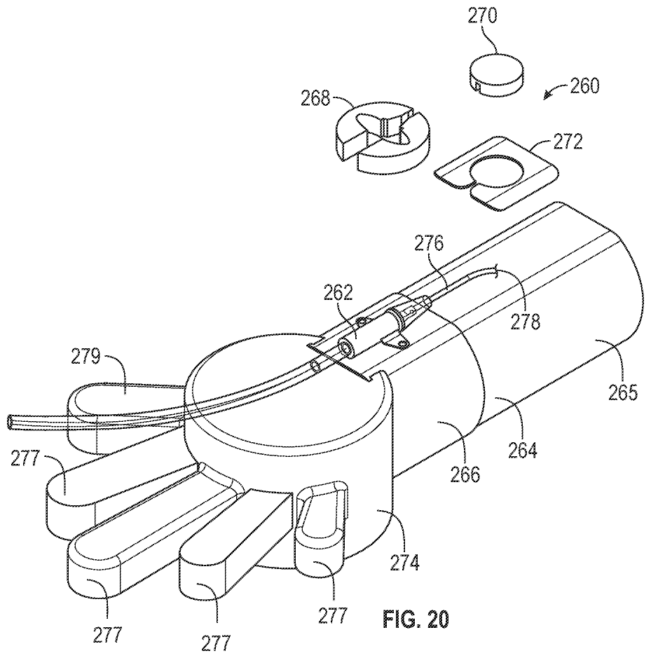

FIG. 19 is a perspective view of wrist band device 260 incorporating microstructures for application of appliance 262 to skin proximate wrist 264. Wrist band device 260 can comprise band 266, die cut or molded foam 268, foam dressing 270 and backing 272. FIG. 20 is an exploded view of wrist band device 260 of FIG. 19 showing band 266 and appliance 262. FIGS. 19 and 20 are discussed concurrently.

Wrist 264 can be located between hand 274 and arm 275. Hand 274 can include fingers 277 and thumb 279.

Band 266 can comprise a band or hoop of material configured to fit around wrist 264. Band 266 can be stretchable to fit over arm 275. Band 266 can additionally include features, such as clasps or buckles, to fit over arm 275. Band 266 can be adjustable to fit over different sized hands and to conform to wrists of different diameters. Band 266 can be an endless loop or a strap having ends fastened to each other.

Appliance 262 can be attached to band 266 via any suitable method, such as adhesive, glue, fasteners, locking mechanism, or friction. Die cut or molded foam 268 can be attached to band 266 to surround appliance 262. Die cut or molded foam 268 can include a cut-out or pocket to fit around the perimeter of appliance 262. Die cut foam can comprise a structure to buffer and pad appliance 262 from external impacts. In examples, appliance 262 can comprise a catheter comprising tube 276 configured to enter wrist 264 at incision 278. In the illustrated example, appliance 262 can comprise a radial arterial catheter. Foam dressing 270 can be attached to backing 272 via adhesive. Foam dressing 270 can be configured to cover incision 278. Foam dressing 270 can be configured to absorb fluid from incision 278 and, as such, can comprise absorbent material such as cotton or gauze. Foam dressing 270 is thus configured to be changed as needed or desired upon becoming soiled. Foam dressing 270 can be an optional feature.

In order to minimize irritation at incision 278 via tube 276, it can be desirable immobilize tube 276. Band 266 can include microstructures to minimize movement of wrist band device 260. In particular, the microstructures can limit axial movement of band 266 along wrist 264 and circumferential rotation of band 266 about wrist 264, which thereby minimizes tugging on tube 276.

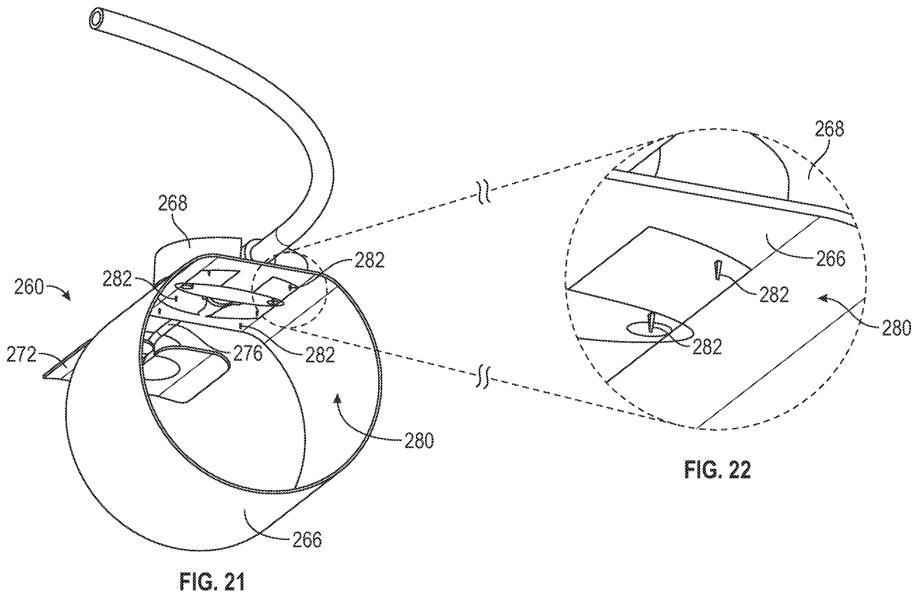

FIG. 21 is a perspective view of interior 280 of wrist band device 260 of FIG. 19 showing microstructures 282. FIG. 22 is a close-up view of a microstructure 282 of FIG. 21. FIGS. 21 and 22 are discussed concurrently. Microstructures 282 can extend into interior 280 to engage tissue of wrist 264 (FIG. 20). Microstructures 282 can comprise any micro-sized structure suitable for grabbing onto or piercing into tissue, e.g., skin, such as barbs, hooks, anchors, needles, blades, fishscales, pillars, hairs (i.e., a microstaple, a microbarb, a microneedle, a microblade, a microanchor, a microhook, a microfishscale, a micropillar, and a microhair) and the like. In examples, microstructures 282 can additionally utilize or be replaced by the microstructures described with reference to FIGS. 16-18. Microstructures 282 can engage skin of wrist 264 to prevent rotation and linear movement along wrist 264, thereby reducing irritation at incision 116 (FIG. 20) from tube 278. In the illustrated example, eight of microstructures 282 are provided on band 266 opposite die cut foam 268 and alongside appliance 262. In examples, different numbers of microstructures 282 can be used. It is desirable to have microstructures 282 on opposite sides of appliance 262 to prevent rotation about the surface of band 266.

FIG. 23 is a perspective view of appliance device 300 configured to attach catheter 302 to tissue using a plurality of microstructure arrays 304A-304D attached to backing 306. Appliance device 300 can further comprise coupler 308, which can comprise one or more of pad 310 and straps 312. Straps 312 can comprise a coupling, an attachment feature, an appliance securing feature, an appliance structure, a coupling device as is used herein.

Backing 306 can be fabricated of any of the materials described herein with respect to other backings. Microstructure arrays 304A-304D are illustrated as comprising four microstructure devices, but can comprise any of the microstructures or microstructure arrays described herein.

Coupler 308 can comprise a coupling, an attachment feature, an appliance securing feature, or an appliance structure, as is used herein. Coupler 308 can be used to indirectly attach catheter 302 to tissue. Coupler 308 can be directly attached to backing 306 and can directly catheter to 302. Pad 310 can be attached to backing 306 by any suitable means, such as adhesive, glue, and fasteners. Straps 312 can be attached to the one or more pads 310 via various means. The illustrated pad 310 is shown on one side of catheter 302. A second instance of pad 310 can be attached to backing 306 on an opposite side of catheter 302. Straps 312 can span pad 310 illustrated and a similar pad on the opposite side of catheter 302. Straps 312 can be fixed to pads 310, such as via adhesive or sutures. In such examples, straps 312 can comprise buckles, hook and loop material or fasteners to provide adjustment to the length of straps 312 extending over catheter 312. In examples, straps 312 can comprise elastic material that can expand to receive catheter 302 and can then retract to compress down on catheter 302 to immobilize catheter 302.

In examples, catheter 302 can comprise a urinary catheter, a peritoneal dialysis catheter, a vascular catheter, an arterial catheter, a hemodialysis catheter, a venous catheter, a central venous catheter, a peripherally inserted central catheter (PICC) and a peripheral intravenous catheter. However, appliance device 300 can be used with other appliances, such as drains, tubes, bags, containers, administering devices, communication devices, sensors, electrodes, monitors and other appliances and devices described herein.

### Definitions and Abbreviations

The terminology used herein is for the purpose of describing particular embodiments only, and is not intended to limit the scope of the present invention, which will be limited only by the appended claims. Unless defined otherwise, all technical and scientific terms used herein have the same meaning as commonly understood by one of ordinary skill in the art to which this invention belongs. As used in the specification and appended claims, unless specified to the contrary, the following terms have the meaning indicated.

As used herein and unless otherwise indicated, the terms “a” and “an” are taken to mean “one”, “at least one” or “one or more”. Unless otherwise required by context, singular terms used herein shall include pluralities and plural terms shall include the singular.

Reference to the term “e.g.” is intended to mean “e.g., but not limited to” and thus it should be understood that whatever follows is merely an example of a particular embodiment, but should in no way be construed as being a limiting example. Unless otherwise indicated, use of “e.g.” is intended to explicitly indicate that other embodiments have been contemplated and are encompassed by the present invention.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” or “some embodiments” or “certain embodiments” mean that a particular feature, structure or characteristic described in connection with the embodiment is included in at least one embodiment of the present invention. Thus, the appearances of the phrases “in one embodiment” or “in an embodiment” or “in certain embodiments” in various places throughout this specification are not necessarily all referring to the same embodiment. Furthermore, the particular features, structures, or characteristics may be combined in any suitable manner in one or more embodiments.

“Optional” or “optionally” means that the subsequently described event of circumstances may or may not occur, and that the description includes instances where said event or circumstance occurs and instances in which it does not.

“Substantially” or “essentially” means nearly totally or completely, for instance, 95% or greater of some given quantity.

As used herein, the term “device” as used generally means a device used for reducing pain or inflammation.

As used herein, the term “tissue” means any human or other animal tissue including, but not limited to skin, muscle, tendon, bone, heart, lung, kidney, brain, bowel, colon, rectum, stomach, esophagus, etc.

Reference to the term “PMMA” as used herein is meant to refer to poly(methyl methacrylate), which is also known as Poly(methyl 2-methylpropenoate (IUPAC name), polymethyl methacrylate, or more commonly known as Plexiglass™.

The terms “affixed” and “attached” are used interchangeably throughout, and have their ordinary meaning, e.g., being connected or fastened to something else. Accordingly, other terms such as “connected”, “fastened”, and “bound” may also be used in a similar manner.

The term “grasp” or “grasping” is used herein, to describe a microstructure-based anchoring of a closure device to its intended location on the surface of the skin or tissue to which it is applied; said anchoring not requiring penetration into the skin or tissue by the microstructures, but instead e.g., being anchored via friction generated by the contact of the microstructures with the skin or tissue. In some embodiments, the device is anchored by grasping, optionally with or without the assistance of the other various components of the present devices and systems, e.g., a protective cover or adhesive.

The term “penetration” or “penetrate” is meant herein to refer to the action of piercing the skin or tissue, e.g., with one or more of the microstructures disclosed herein.

The term “inflammation” is meant to have its ordinary medical meaning, i.e. a biological response of a tissue to a harmful stimulus. Common signs of inflammation include pain, heat, redness (erythema), swelling (edema), and loss of function.

The term “base” is meant generally to describe a supporting means from which one or more microstructures protrude. In some embodiments, the base comprises a plurality of microstructures; and in other embodiments devices comprising singular microstructures on a base are provided. The base may be a separate component upon which one or more microstructures are affixed; or alternatively, the microstructures and the base may be one continuous component that are fabricated at the same time, optionally from the same or different materials. For example, but not to be limited in any way, some embodiments of the present invention provide for devices comprising one or more microstructure arrays patterned on a base, wherein both the base and the microstructures are made out of polymethylmethacrylate (PMMA) or a metal such as stainless steel. In one such embodiment, the microstructures are manufactured using a replica molding technique, wherein both the microstructures and the array are manufactured simultaneously, and are thus in essence one single component. Further embodiments provide for a variety of base specifications including, e.g., thickness, length, width, and composition. In certain embodiments, the base comprises a substantially planar upper surface and a substantially planar lower surface; said upper surface comprising one or more microstructures, and said lower surface optionally being affixed to a backing. In such an embodiment, the upper surface comprising the microstructures is intended to be put in contact with the skin or tissue of the patient and the lower surface is intended to be exposed to the external environment, or optionally to be in contact with a protective cover, e.g., a cover comprising adhesive.

The terms “array” and “microstructure array” are used herein to describe a two-dimensional configuration of two or more microstructures on a “base”, as described herein, said base having a substantially planar upper surface from which the microstructures protrude. The “array” may be in any suitable shape or pattern, and the array may be of any suitable size or dimensions. Furthermore, arrays may comprise any suitable number or density of microstructures, said microstructures optionally extending from the base at angle, or in a substantially perpendicular manner.

An “array region” as used herein is meant to describe an area of the present devices upon which one or more microstructure arrays are affixed. Accordingly, in some embodiments the array region is a portion of the backing upon which one or more bases are affixed, said bases each comprising one or more microstructure or microstructure arrays. In some particular embodiments, the devices of the present invention comprise at least two “array regions” that are separated from one another by an isthmus, as described herein.

The term “isthmus” as used herein refers to a space with no arrays, that separates two or more microstructure “arrays” or “array regions”. “Isthmus separation” refers to the distance separating two arrays on opposing sides of an isthmus. The isthmus may comprise any suitable material, and may in some embodiments be rigid, flexible, and/or stretchable. The size and shape of the isthmus may vary, and in some embodiments the device will comprise an isthmus and a backing, both being made out of the same material, while in other embodiments the material comprised in the isthmus will differ from that of the backing. In certain embodiments, the isthmus is simply created by affixing two or more microstructure arrays upon a backing such that a space separates the two arrays. In still other embodiments, the isthmus is a portion of a base comprising a plurality of microstructure arrays (i.e., the isthmus and the microstructures are made of the same material). Non-limiting examples of two different types of isthmuses can be seen in FIGS. 17 and 18 wherein the shape, composition (silicone vs. thermoplastic polyurethane (“TPU”)), and properties (i.e. stretchable vs. non-stretchable) have been varied. In some embodiments, the isthmus ranges from 1 mm in length to 15 mm in length. Accordingly, in these embodiments, the devices of the present invention may comprise isthmuses that are 1 mm in length, or they may comprise isthmuses that are 2 mm; 3 mm; 4 mm; 5 mm; 6 mm; 7 mm; 8 mm; 9 mm; 10 mm; 11 mm; 12 mm; 13 mm, 14 mm; or 15 mm in length, including all decimals (e.g., 1.5 mm, 1.6 mm, 1.7 mm, etc.) and ranges (e.g., 1-15 mm, 5-10 mm, 10-15 mm, 3-4 mm, 5-6 mm, 6-8 mm, etc.) in between, of the isthmus lengths set forth herein. The width of the isthmus may vary. In some embodiments the isthmus width is the same as the base or backing of the device. In other embodiments, the isthmus is wider or narrower than the base or backing of the device. Thus, the width of the isthmus may range from as small as 1 mm to as large as 50 cm or more.

Accordingly, isthmus widths may range from approximately 1 mm, 2 mm, 3 mm, 4 mm, 5 mm, 6 mm, 7 mm, 8 mm, 9 mm, 1 cm, 2 cm, 3 cm, 4 cm, 5 cm, 10 cm, 20 cm, 30 cm 40 cm 50 cm or longer, including all integers (e.g., 11 mm, 12 mm 13 mm, etc.) and ranges (e.g., 2 mm-50 cm, 5 mm-15 mm, 5 mm-10 mm, etc.) in between of the isthmus widths set forth herein.

As used herein, when components are said to be positioned or distributed “anisotropically”, it is meant that the components are not uniform throughout, but instead their properties vary directionally. Thus, e.g., in some embodiments, anisotropic positioning refers to variation in the components of individual microstructures comprised in a microstructure array, said microstructures comprising directional variability in their physical properties, e.g., their aspect ratios or angles of attachment to a backing. In other embodiments, this variability may be in regard to directional differences between different arrays. Anisotropic variability may be in one direction, or in more than one direction.

As used herein, the term “microstructure” refers to a three-dimensional structure projecting from or connected to a base. A microstructure may be an integral part of the base (i.e., the microstructure and base are monolithic). Alternatively, the microstructure may be of separate construction than the base but be joined to the base (e.g., through adhesive, bonding, etc.).

Microstructures typically have dimensions on the micron size scale, although certain dimensions may extend into the millimeter size scale (e.g., length or height) and certain dimensions may be smaller than one micron (e.g., nano scale tip width). Representative microstructures include microneedles, microblades, microanchors, microfishscale, micropillars, and microhairs.

A microstructure includes a foundation, a tip, and a body joining the foundation with the tip.

As used herein, the term “foundation” refers to the two-dimensional area where the base meets the microstructure. The foundation can be any two-dimensional shape, including a circle, oval, ellipse, triangle, rectangle, square, quadrilateral, or higher-order polygon.

As used herein, the term “tip” refers to the end of the microstructure distal to the foundation and base. The tip may be a single point (e.g., a needle), a line (e.g., a blade), or other shape.

As used herein, the term “body” refers to the portion of the microstructure between the foundation and the tip. The body may also be referred to herein as a “shaft” of the microstructure. The body has a “length” that is equal to the longest distance connecting a point on the foundation to the tip.

The microstructure can be either straight or curved. In certain embodiments, the body connects the foundation to the tip without curvature along its length. In other embodiments, the body is curved along its length between the foundation and the tip.

As used herein, the term “straight” refers to a microstructure having no curvature (i.e., no concave or convex surfaces) along the body between the foundation and the tip. As used herein, the term “curved” refers to a microstructure having one or more concave or convex surfaces along the body between the foundation and the tip.

Straight and curved microstructures can be defined in terms of a “face angle” (θF), which is the smallest angle formed between the base and the microstructure. The face angle will always be greater than the structure angle.

As used herein, the term “articulated” refers to a microstructure that does not curve continuously but instead curves via one or more joints connecting straight portions. An articulated microstructure may also be referred to as “beveled.”

As used herein, the term “convex” refers to a microstructure having at least one line along the outer surface of the body that deviates outwardly from a straight line between the foundation and the tip.

As used herein, the term “concave” refers to a microstructure having at least one line along the outer surface of the body that deviates inwardly from a straight line between the foundation and the tip.

As used herein, the term “angled” refers to a microstructure that is not perpendicular to the base. The angle of a microstructure in relation to the base can be understood with reference to The “center point” is the center of the foundation. The angle (“center point angle”; θc) formed between the line and the base defines the angle of the entire microstructure. For microstructures, if the tip is not directly above the center point then the microstructure is angled.

Curved microstructures may be defined by an angle if a tip-to-center point line can be drawn so as to define an angle in relation to the base. However, extensively curved microstructures may not allow a straight line to be drawn through the body from the tip to the center point. As used herein, the term “microneedle” is intended to refer to any microstructure comprising straight or tapered shafts. In one embodiment, the diameter of the microneedle is greatest at the base end of the microneedle and tapers to a point at the end distal the base. The microneedle can also be fabricated to have a shaft that includes both a straight (untapered) portion and a tapered portion. The microneedles can be formed with shafts that have a circular cross-section in the perpendicular, or the cross-section can be non-circular. For example, the cross-section of the microneedle can be polygonal (e.g. star-shaped, square, rectangular, and triangular), oblong, or another shape. The tip portion of the microneedles can have a variety of configurations. The tips can be symmetrical or asymmetrical about the longitudinal axis of the microneedle shaft. In one embodiment, the tips are beveled. In another embodiment, the tip portion is tapered. In one embodiment, the tapered tip portion is in the shape of a pyramid on a shaft portion having a square cross-section, such that the microneedle is in the shape of an obelisk. Of course, the tip and/or shaft can be rounded, or have another shape, as well. In some embodiments the microneedles comprise a shape that is a e.g., rod, cone, square, rectangle, pyramid, cylinder.

As used herein, the term “microblade” is intended to refer to a needle-like microstructure comprising a tip that is not a point, but is instead a blade. The tip portion of these structures is wide in a first dimension (50 μm in this picture) and very narrow in a second dimension, with respect to the first dimension (e.g., less than 10 μm in this picture). Furthermore, in some embodiments, the thickness at the tip is smaller than the width of the microblades near their base.

As used herein, the term “microanchor” is intended to refer to any microstructure capable of anchoring a device according to the present disclosure to skin or tissue. Examples of microanchors include microstructures with ends shaped like hooks or barbs. As used herein, the term “barb” refers to a tip configuration comprising angled portions projecting away from the tip in order to secure the barb within the penetrated skin or tissue.

As used herein, the term “microfishscale” is intended to refer to any microstructure comprising a scale that partially overlaps, with other scales of microscale dimensions and mimics the scale of a fish.

As used herein, the term “micropillar” is intended to refer to any microstructure comprising a cylindrical shape.

As used herein, the term “microhair” is intended to refer to any microstructure comprising hair-like features which enable the contacting and sticking of the microhair to another object via van der Waals forces.

As used herein, the term “Microstaple” is intended to refer to the product, microMend®, which is manufactured by KitoTech Medical (Seattle, Wash.).

As used herein, the term “microMend” refers to the wound closure product designated MM12MP2 (12 packages of multipack of 2 MP devices per package in a box), MM12MP4 (12 packages of multipack of 4 MP devices per package in a box), MM12SM2 (12 packages of multipack of 2 SMALL devices per package in a box), MM12SM4 (12 packages of multipack of 4 SMALL devices per package in a box), MM12XS2 (12 packages of multipack of 2 EXTRA SMALL devices per package in a box), MM12W2S (12 packages of multipack of 2 WIDE-S devices per package in a box), MM12W2A (12 packages of multipack of 2 WIDE-A devices per package in a box), MM12LG2 (12 packages of multipack of 2 LARGE devices per package in a box), MM12S2S (Wide-s, 12 packs of 2) or SU2 (Umbilical device, pack of 2), which are manufactured by KitoTech Medical (Seattle, Wash.). Information on the product can be found at the website: www.micromendskinclosure.com.

As used herein, the term “patch” refers to a piece of material that is worn on the skin or other tissue.

The term “tapered” is meant to describe a microstructure wherein the width or diameter gradually diminishes along the length of the needle from the base to the tip, such that the base comprises the largest width or diameter, and the tip comprises the smallest width or diameter. A “partially tapered” microstructure is one in which a portion of the microstructure is tapered and a portion of the microstructure is not tapered. For example, but not to be limited, such a microstructure can comprise a tapered portion extending from a block shaped base; or e.g., a cylindrical base portion can extend toward the tip for a certain length, and then a tapered portion can continue to the tip. Alternatively, the microstructure can comprise a tapered portion extending from the base, with a non-tapered portion being at the tip end of the microstructure.

The term “stretchable” as used herein is meant to encompass any material that can be elongated in any direction, e.g., as a result of a pulling force. “Stretchable” encompasses the term “elastic” and thus an object that is said to be stretchable should be understood to optionally comprise elasticity. Thus in some embodiments, if an object is said to be stretched, this is meant to include at least two embodiments; the first being that the stretching force will be counteracted by a retractile force, and thus once the stretching force is removed, the object will inherently attempt to retract (e.g., as is the case with an elastic object). The second embodiment is one in which the object does not inherently comprise elasticity, and thus no such retractile force is inherent.

The term “flexible” is meant to describe any material that is capable of sustaining a bending force without being damaged. In some embodiments, a “flexible” material comprises enough flexibility as to allow the device of the present invention to bend so as to fit the contours of the biological barrier, such as, e.g., the skin, vessel walls, or the eye, to which the device is applied.

The term “backing” as used herein, is meant to describe an optional component of the present devices which is attached to one or more arrays. In some embodiments the backing attaches two or more microstructure arrays together. As is thoroughly described in the detailed description, the backing may comprise any suitable material, and in several embodiments, it is flexible, stretchable, elastic, or combinations thereof.

The term “cover” as used herein in meant to describe an optional component of the systems disclosed herein whereby it covers the device. After application of the devices of the present invention, such a cover may be optionally applied over and/or attached to the top of the device, e.g., assist in securing the device in place. The covers may be made of any suitable material, as is discussed and defined thoroughly in the detailed description section below. In some embodiments the covers comprise adhesive.

When it is said that one or more microstructures are “affixed to a backing” it is meant that the microstructures may optionally be either directly affixed to the backing, or indirectly affixed to the backing (e.g., in some embodiments, “affixed to a backing” is meant to encompass the scenario wherein the microstructures are fashioned on, or affixed to, a base, said base being affixed to a backing). Accordingly, the phrase “one or more microstructures affixed to a backing” can appropriately be used interchangeably with the phrase “a backing comprising one or more microstructures.”

As used herein, the term “pitch” is meant to describe the distance between the tips of two or more adjacent microstructures in a given array, or in two or more separate arrays. In some embodiments the pitch ranges from 30 μm to 1 cm or more. Accordingly, certain embodiments provide for microstructure arrays as disclosed herein, wherein the microstructures are separated from one another with a pitch of 30 μm, 50 μm, 70 μm, 90 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, or more, including all decimals (e.g., 3.1 mm, 3.2 mm, 3.3 mm, etc.) and ranges (e.g., 1-10 mm, 5-10 mm, 7-10 mm, etc.) in between, of the microstructure array pitches set forth herein. The pitch may be constant throughout an array, e.g., an equal distance separates all microstructure tips from one another in a given array; or the pitch may vary.

Reference herein to the term “tape” or “microstructure tape” or “microstructure array tape”, is simply meant to describe an adhesive-comprising microstructure array roll bandage, as described herein.

The term “applicator” as used herein is meant to describe any machine or instrument that is used to affix a device, e.g., to the skin, or the use of medical instruments such as forceps, tweezers, clamps, pins, etc. to apply such a device would be considered to be use of an applicator. The term “applicator” also refers to the roll on hand-held dispenser disclosed herein. Thus, when it is said that the device is applied without an applicator, this is to be understood as being applied by human hand, without the aid of a machine or instrument.

### Example Appliance Attachment Devices

1. An attachment device used to attach appliances to tissue that incorporates microstructures.
2. An attachment device according to claim 1, in which the microstructures are microstaples.
3. An attachment device according to claim 1, in which the tissue is skin.
4. An attachment device according to claim 3, in which the device is used to attach appliances to remove fluids or blood from the body.
5. An attachment device according to claim 4, in which the appliances comprise catheters, drains, tubes, or ostomy bags.
6. An attachment device according to claim 5, in which the appliance is an ostomy bag.
7. An attachment device according to claim 3, in which the appliances are used for diagnosis or monitoring of a medical condition.
8. An attachment device according to claim 7, in which the appliance is a glucose monitor, EEG monitor or an EKG monitor.
9. An attachment device according to claim 5, in which the appliance is a catheter.
10. An attachment device according to claim 9, in which the appliance is used for dialysis.
11. An attachment device according to claim 3, in which the appliance is used to administer nutrients, drugs or fluids or blood.
10. An attachment device according to claim 9, in which the appliance is used to administer insulin or chemotherapy drugs.
13. An attachment device according to claim 1, in which attachment to the appliance of the tissue is achieved via direct incorporation of the microstructures into the appliance.
14. An attachment device according to claim 1, in which attachment to the appliance of the tissue is achieved indirectly via sutures, staples, tapes, or adhesives.
15. An attachment device according to claim 1, used in an animal.
16. An attachment device according to claim 1, used in a human.
17. An attachment device containing microstructures that is not attached to the appliance and used to secure the appliance on the tissue.
18. An attachment device according to claim 17, in which the microstructures are microstaples.
19. An attachment device according to claim 17, in which the tissue is skin.
20. An attachment device according to claim 17, in which the attachment device comprises an attachment feature to couple to an appliance.
21. An attachment device according to claim 19, in which the attachment feature comprises a flange.
22. An attachment device according to claim 19, wherein the attachment feature comprises a ring.
23. An attachment device according to claim 19, wherein the attachment device comprises a hole to allow for passage of a tube, wire, probe, nutrient, or drug.
24. A device according to any preceding claim wherein the attachment device comprises:

a base layer;

a coupling attached to an air side of the base layer; and

a microstructure attached to a skin side of the base layer.

25. A device according to claim 24, further comprising:

an adhesive layer coupling the microstructure to the skin side of the base layer; and

a release liner covering the adhesive layer and the microstructure.

26. A device according to claim 25, further comprising a moisture barrier extending across the base layer.
27. A device according to claim 24, wherein the attachment device and the coupling are circular.

### Example Coupling Devices

1. A coupling device for attaching an appliance to a patient, the coupling device comprising.

a base layer;

a coupling attached to an air side of the base layer; and

a microstructure attached to a skin side of the base layer.

2. The coupling device according to claim 1, further comprising:

an adhesive layer coupling the microstructure to the skin side of the base layer; and

a release liner covering the adhesive layer and the microstructure.

3. The coupling device according to claim 2, further comprising a moisture barrier extending across the base layer.
4. The coupling device according to claim 1, further comprising a patient interaction feature.
5. The coupling device according to claim 4, wherein the coupling device and the coupling are circular, and the patient interaction feature is within a perimeter of the coupling.
6. The coupling device according to claim 4, wherein the patient interaction feature comprises a fluid conduit.
7. The coupling device according to claim 6, wherein the fluid conduit comprises a hole.
8. The coupling device according to claim 4, wherein the patient interaction feature comprises a sensor.
9. The coupling device according to claim 8, wherein the sensor comprises an EKG electrode.
10. The coupling device according to claim 8, wherein the sensor comprises a glucose monitor.
11. The coupling device according to claim 4, wherein the patient interaction feature comprises a hydrogel.
12. The coupling device according to claim 1, wherein the coupling comprises a flange, a threaded coupling, or hook and loop fastener material.
13. The coupling device according to claim 1, wherein the coupling comprises sutures, staples, tapes or adhesives.
14. The coupling device according to claim 1, further comprising an appliance coupled to the coupling.

### Example Medical Devices

1. A medical device system comprising:

a coupling device comprising:
a base layer;
a coupling attached to an air side of the base layer;
a microstructure attached to a skin side of the base layer; and
a patient interaction feature; and

an appliance comprising:
a structure mounted to the coupling to interface with the patient interaction feature.
   2. The medical device system of claim 1, wherein the appliance comprises an ostomy bag.
   3. The medical device system of claim 2, wherein the patient interaction feature comprises a fluid conduit.
   4. The medical device system of claim 3, wherein the fluid conduit comprises a hole.
   5. The medical device system of claim 1, wherein the patient interaction feature comprises a sensor.
   6. The medical device system of claim 5, wherein the appliance comprises an EKG electrode.
   7. The medical device system of claim 5, wherein the appliance comprises a glucose monitor.
   8. The medical device system of claim 1, wherein the patient interaction feature comprises a hydrogel.
   9. The medical device system of claim 1, wherein the appliance comprises a catheter.

### Example Appliance Attachment Devices

1. An appliance device comprising:

a base layer;

an appliance structure attached to an air side of the base layer; and

a microstructure attached to a skin side of the base layer.

2. The appliance device of claim 1, wherein the appliance structure comprises a ventilatory face mask or endotracheal tubing.
3. The appliance device of claim 1, further comprising a patient interaction feature on the base layer to interface with the appliance structure.
4. The appliance device of claim 3, wherein the patient interaction feature comprises a sensor.
5. The appliance device of claim 4, wherein the appliance structure comprises an EKG electrode.
6. The appliance device of claim 3, wherein the appliance structure comprises a monitor.
7. The appliance device of claim 6, wherein the monitor comprises a glucose monitor.
8. The appliance device of claim 6, wherein the monitor comprises an EKG monitor.
9. The appliance device of claim 6, wherein the monitor comprises an EEG monitor.
10. The appliance device of claim 3, wherein the patient interaction feature comprises a hole.
11. The appliance device of claim 10, wherein the appliance structure comprises an ostomy bag.
12. The appliance device of claim 10, wherein the appliance structure comprises a drain.
13. The appliance device claim 12, wherein the appliance structure comprises a surgical drain.
14. The appliance device of claim 10, wherein the appliance structure comprises a tube.
15. The appliance device of claim 14, wherein the appliance structure comprises an endotracheal tube.
16. The appliance device of claim 14, wherein the appliance structure comprises a chest tube.
17. The appliance device of claim 14, wherein the appliance structure comprises a nasogastric tube.
18. The appliance device of claim 14, wherein the appliance structure comprises an enterostomy tube.
19. The appliance device of claim 10, wherein the appliance structure comprises a catheter.
20. The appliance device of claim 19, wherein the appliance structure comprises a urinary catheter.
21. The appliance device of claim 19, wherein the appliance structure comprises a peritoneal dialysis catheter.
22. The appliance device of claim 10, wherein the appliance structure comprises a vascular catheter.
23. The appliance device of claim 22, wherein the appliance structure comprises an arterial catheter.
24. The appliance device of claim 22, wherein the appliance structure comprises a hemodialysis catheter.
25. The appliance device system of claim 22, wherein the appliance structure comprises a venous catheter.
26. The appliance device of claim 25, wherein the appliance structure comprises a central venous catheter.
27. The appliance device of claim 25, wherein the appliance structure comprises a peripherally inserted central catheter (PICC).
28. The appliance device of claim 25, wherein the appliance structure comprises a peripheral intravenous catheter.
29. The appliance device of claim 1, wherein the appliance structure is configured to secure more than one appliance.
30. The appliance device of claim 1, further comprising sutures, staples, tapes, or bandages to facilitate attachment of the base layer to skin.
31. The appliance device of claim 30, further comprising sutures configured to facilitate attachment of the base layer to skin.
32. The appliance device of claim 30, further comprising staples configured to facilitate attachment of the base layer to skin.
33. The appliance device of claim 30, further comprising tape configured to facilitate attachment of the base layer to skin.

### Example Appliance Attachment Device System

1. An appliance attachment system comprising.
an appliance;
a first component comprising an adhesive backing containing microstructures that are configured to attach to skin; and
a second component configured to attach the appliance to the first component.
   2. The appliance attachment system of claim 1, wherein the second component is incorporated into the first component.
   3. The appliance attachment system of claim 1, wherein the first and second components are provided individually and configured to be subsequently attached to one another.
   4. The appliance attachment system of claim 3, wherein the first and second components are each provided individually in a kit.
   5. The appliance attachment system of claim 1, wherein the first component can be attached to the skin and then the second component attached to the first component.
   6. The appliance attachment system of claim 1, wherein the first component is configured to be attached to the second component before the first component is attached to the skin.
   7. The appliance attachment system of claim 1, wherein a catheter can be attached to the second component before the first component is attachment to the skin.
   8. The appliance attachment system of claim 1, wherein a catheter can be attached to the second component after the first component is attached to the skin.
   9. The appliance attachment system of claim 1, wherein the first component comprises a cover comprising an adhesive backing containing microstructures.

### Example Appliance Attachment Covers

1. An appliance attachment cover comprising:

a cover body;

an appliance securing feature incorporated into the cover body; and

a microstructure attached to a skin side of the cover body.

2. The appliance attachment cover of claim 1, wherein the appliance securing feature is configured to engage a sensor or a monitor.
3. The appliance attachment cover of claim 1, wherein the appliance securing feature is configured to engage a surgical drain.
4. The appliance attachment cover of claim 1, wherein the appliance securing feature is configured to engage a drain.
5. The appliance attachment cover of claim 1, wherein the appliance securing feature is configured to engage a tube.
6. The appliance attachment cover of claim 1, wherein the appliance securing feature is configured to engage a catheter.
7. The appliance attachment device of claim 6, further comprising a catheter configured to engage the appliance securing feature.
8. The appliance attachment device of claim 6, wherein the appliance securing feature comprises a cavity molded to a shape of a catheter.
9. The appliance attachment device of claim 8, wherein the appliance securing feature is configured to conform to different sized catheters.
10. The appliance attachment device of claim 6, wherein the appliance securing feature is configured to engage a plurality of catheters.
11. The appliance attachment cover of claim 1, wherein the appliance securing feature is configured to engage an arterial catheter.
12. The appliance attachment device of claim 11, further comprising an arterial catheter.
13. The appliance attachment device of claim 11, wherein the appliance securing feature comprises a cavity molded to a shape of an arterial catheter.
14. The appliance attachment device of claim 11, wherein the appliance securing feature is configured to engage a plurality of arterial catheters.
15. The appliance attachment cover of claim 1, wherein the cover body includes a feature for attaching to an appliance.
14. The appliance attachment cover of claim 1, further comprising an adhesive backing containing microstructures that is incorporated into the cover.
17. The appliance attachment device of claim 14, wherein the cover and the adhesive backing with microstructures are incorporated into a single securement device.
18. The appliance attachment device of claim 14, further comprising a component configured to attach directly to an appliance.
19. The appliance attachment device of claim 18, wherein the component and the cover are provided individually in a kit.
20. The medical device of claim 18, wherein the cover comprises a molded piece configured to conform to a shape of the component.
21. The medical device of claim 20, wherein the component is attached directly to a catheter.
22. The medical device of claim 20, wherein the component is attached directly to a vascular catheter.
23. The medical device of claim 20, wherein the component is not attached to a catheter.
24. The medical device of claim 23, wherein the component is indirectly attached to a catheter.
25. The medical device of claim 20, wherein the molded piece is able to conform to different sized catheter attachment devices.
26. The medical device of claim 25, wherein the molded piece contains foam material to enable conformability to different sized catheter attachment devices.
27. The medical device of claim 25, wherein the molded piece contains foam material to enable conformability to different sized vascular catheter attachment devices.
28. The medical device of claim 18, wherein the molded piece and cover form a catheter securement device.
29. The medical device of claim 28, wherein the molded piece and cover are each provided individually as a securement device kit.
30. The medical device of claim 1, wherein the appliance securing feature, the cover and a molded piece comprise a catheter securement device.
31. The medical device of claim 30, wherein the appliance securing feature, the cover and the molded piece are provided individually in a kit.
32. The medical device of claim 1, wherein the appliance securing feature is configured to attach to more than one catheter.
33. The medical device of claim 6, wherein an arm band is attached directly or indirectly attached to the catheter securement device.
34. The medical device of claim 12, wherein an arm band is attached directly or indirectly attached to the vascular catheter securement device.
35. The medical device of claim 6, wherein the securement device includes a first component that is directly or indirectly attached to the catheter and a second component that covers the port site where the catheter enters the skin.
36. The medical device of claim 35, wherein the second component is a dressing.
37. The medical device of claim 36, wherein the second component contains an anti-septic or anti-microbial agent.
38. The medical device of claim 37, wherein the agent is chlorhexidine.
39. The medical device of claim 35, wherein the cover of the port site contains microstructures.
40. The medical device of claim 35, wherein the first component and the second component comprise one device.
41. The medical device of claim 35, wherein the first component and the second component are separate components of the vascular catheter device.
42. The medical device of claim 35, wherein, the first component and the second component are provided individually in a kit.
43. The medical device of claim 35, wherein the vascular catheter securement device includes a cover over the first and second components.

### Example Radial Arterial Catheter Attachment Devices

1. A medical device configured for attaching a radial arterial catheter to a patient, the appliance attachment device comprising:
a base layer;
an appliance structure attached to an air side of the base layer, the appliance structure configured to engage a radial arterial catheter; and
a microstructure attached to a skin side of the base layer.
   2. The medical device of claim 1, wherein the base layer comprises a wrist band that is configured to encircle a distal part of an extremity just proximal to a wrist joint.
   3. The medical device of claim 2, wherein the wrist band is configured to fully encircle a circumference of the wrist.
   4. The medical device of claim 2, wherein the appliance structure and the wrist band are combined into a single securement device.
   5. The medical device of claim 2, wherein the appliance structure and the wrist band are separated into individual components.
   6. The medical device of claim 2, wherein the appliance structure and the wrist band are each provided individually in a kit.
   7. The medical device of claim 2, wherein the wrist band contains microstructures.
   8. The medical device of claim 2, wherein the wrist band contains adhesive.
   9. The medical device of claim 2, wherein the wrist band does not contain adhesive.
   10. The medical device of claim 2, wherein the microstructure is attached to an elastic backing enabling attachment to remain in place with movement or edema in an area of the wrist.
   11. The medical device of claim 2, wherein the wrist band is adjustable so that it can be fitted to wrists of different circumferences.

### Example Appliance Attachment Devices Methods

1. A method for coupling an appliance to a patient, the method comprising:

positioning a backing of a coupling device adjacent tissue;

attaching a microstructure mounted to the backing to the tissue; and

attaching an appliance to a coupling feature of the backing.

2. The method of claim 1, wherein attaching the microstructure mounted to the backing to the tissue comprises adhering an adhesive to the tissue.
3. The method of claim 2, further comprising removing a release liner from the backing.
4. The method of claim 1, further comprising engaging a patient interaction feature of the backing with the tissue.
5. The method of claim 4, wherein engaging the patient interaction feature of the backing with the tissue comprises engaging a sensor with the tissue.
5. The method of claim 4, wherein engaging the patient interaction feature of the backing with the tissue comprises engaging a fluid conduit with the tissue.
6. The method of claim 4, wherein engaging the patient interaction feature of the backing with the tissue comprises engaging a sensor with the tissue.
7. The method of claim 1, wherein attaching an appliance to a coupling feature of the backing attaching an EKG electrode to the coupling feature.
8. The method of claim 1, wherein attaching an appliance to a coupling feature of the backing comprises attaching a glucose monitor to the coupling feature.
9. The method of claim 1, wherein attaching an appliance to a coupling feature of the backing attaching a catheter to the coupling feature.

### Example Appliance Attachment Devices Methods

1. A method for coupling an appliance to a patient, the method comprising:

positioning a backing of an appliance device adjacent tissue;

attaching a microstructure mounted to the backing to the tissue; and

engaging an appliance secured to the patient via the backing with the tissue.

2. The method of claim 1, wherein attaching the microstructure mounted to the backing to the tissue comprises adhering an adhesive to the tissue.
3. The method of claim 2, further comprising removing a release liner from the backing.
4. The method of claim 1, further comprising engaging a patient interaction feature of the backing with the tissue and the appliance.
5. The method of claim 1, wherein engaging an appliance mounted to the backing with the tissue comprises engaging a sensor of an EKG electrode with the tissue.
6. The method of claim 1, wherein engaging an appliance mounted to the backing with the tissue comprises engaging a sensor of an EEG electrode with the tissue.
7. The method of claim 1, wherein engaging an appliance mounted to the backing with the tissue comprises engaging a sensor of a glucose monitor with the tissue.
8. The method of claim 1, wherein engaging an appliance mounted to the backing with the tissue comprises engaging a catheter with the tissue.

1. An appliance attachment cover for holding an appliance against skin, the appliance attachment cover comprising:
a cover body;
an appliance securing feature incorporated into the cover body; and
a microstructure attached to a skin side of the cover body.

2. The appliance attachment cover of claim 1, wherein the appliance securing feature is configured to engage a sensor or a monitor.

3. The appliance attachment cover of claim 1, wherein the appliance securing feature is configured to engage a surgical drain or a tube.

4. The appliance attachment cover of claim 1, wherein the appliance securing feature is configured to engage a catheter.

5. The appliance attachment cover of claim 4, further comprising a catheter configured to engage the appliance securing feature.

6. The appliance attachment cover of claim 4, wherein the appliance securing feature comprises a cavity molded to a shape of a catheter.

7. The appliance attachment cover of claim 4, wherein the appliance securing feature is configured to conform to different sized catheters.

8. The appliance attachment cover of claim 7, wherein the appliance securing feature contains foam material to enable conformability to different sized catheters or catheter attachment devices.

9. The appliance attachment cover of claim 4, wherein the appliance securing feature is configured to engage a plurality of catheters.

10. The appliance attachment cover of claim 1, wherein the cover body includes a feature for attaching to an appliance.

11. The appliance attachment cover of claim 1, further comprising an adhesive backing containing microstructures that is incorporated into the cover.

12. The appliance attachment cover of claim 11, wherein the cover and the adhesive backing with microstructures are incorporated into a single securement device.

13. The appliance attachment cover of claim 11, wherein the cover and adhesive backing are each provided individually as a securement device kit.

14. The appliance attachment cover of claim 4, wherein the appliance securing feature comprises an arm band.

15. The appliance attachment cover of claim 14, wherein the arm band comprises a first component that can be directly or indirectly attached to the catheter and a second component that covers a port site where the catheter enters the skin.

16. The appliance attachment cover of claim 15, wherein the second component is a dressing.

17. The appliance attachment cover of claim 16, wherein the second component contains an anti-septic or anti-microbial agent.

18. The appliance attachment cover of claim 15, wherein the cover of the port site contains microstructures.

19. The appliance attachment cover of claim 15, wherein the first component and the second component comprise one device.

20. The appliance attachment cover of claim 15, wherein the first component and the second component are separate components provided individually in a kit.

21. A method for coupling an appliance to a patient, the method comprising:
positioning a backing of an appliance device adjacent tissue;
attaching a microstructure mounted to the backing to the tissue; and
engaging an appliance secured to the patient via the backing with the tissue.

22. The method of claim 21, wherein attaching the microstructure mounted to the backing to the tissue comprises adhering an adhesive to the tissue.

23. The method of claim 22, further comprising removing a release liner from the backing.

24. The method of claim 21, further comprising engaging a patient interaction feature of the backing with the tissue and the appliance.

25. The method of claim 21, wherein engaging an appliance mounted to the backing with the tissue comprises engaging a sensor of an EKG electrode with the tissue.

26. The method of claim 21, wherein engaging an appliance mounted to the backing with the tissue comprises engaging a sensor of an EEG electrode with the tissue.

27. The method of claim 21, wherein engaging an appliance mounted to the backing with the tissue comprises engaging a sensor of a glucose monitor with the tissue.

28. The method of claim 21, wherein engaging an appliance mounted to the backing with the tissue comprises engaging a catheter with the tissue.

29. The method of claim 21, wherein positioning a backing of an appliance device adjacent tissue comprises positioning a band around an arm of a patient.

30. The method of claim 29, wherein positioning the band around the arm of the patient comprise comprises positioning a first component against a catheter and a second component against an entry site for the catheter.
